(19) United States
(12) Patent Application Publication
(10) Pub. No.: US 2009/0311772 A1
(43) Pub. Date: Dec. 17, 2009

(54) MICRO REFINERY FOR ETHANOL PRODUCTION

(75) Inventor: Thomas Quinn, Los Gatos, CA (US)

Correspondence Address: DERGOSITS & NOAH LLP, Three Embarcadero Center, Suite 410, SAN FRANCISCO, CA, 94111, US

(73) Assignee: E-FUEL CORPORATION, Los Gatos, CA (US)

(21) Appl. No.: 12/488,558

(22) Filed: Jul. 22, 2009

Related U.S. Application Data
(63) Continuation-in-part of application No. 12/110,158, filed on Apr. 25, 2008.
Application No. 12/110,158 is a continuation-in-part of application No. 12/110,242, filed on Apr. 25, 2008.

(51) Int. Cl. C12M 1/36; C12M 1/00

(52) U.S. Cl. 435/286.5; 435/297.1; 435/286.1

(57) ABSTRACT

A micro refinery system includes a fermentation tank, a distillation tube and a membrane ethanol separation mechanism. A batch that includes sugar, yeast and water is mixed in the fermentation tank. Sensors detect the physical characteristics of the batch and the operating status of the system. The sensors are coupled to a control system that compares the detected processing information to a look up table to determine if the system is operating under optimum ethanol productivity conditions. If the sensors detect a problem with the batch, the control system can add chemical components to the batch to correct the chemical imbalance. The system can also facilitate remote operations by transmitting sensed information to an operator computer and receiving control commands from the operator computer.

### CROSS REFERENCE TO RELATED APPLICATIONS

This application is a continuation-in-part of U.S. patent application Ser. No. 12/110,242, “Method For Using Carbon Credits With Micro Refineries” filed on Apr. 25, 2008 and a continuation-in-part of U.S. patent application Ser. No. 12/110,158, “Micro Refinery System For Ethanol Production” filed on Apr. 25, 2008 which are both incorporated by reference.

### FIELD OF INVENTION

The present invention relates generally to a system and method for remotely monitoring micro refinery ethanol systems that convert feedstock and/or discarded alcoholic liquids into ethanol and processing data from the micro refinery systems.

### BACKGROUND OF THE INVENTION

Ethanol fermentation is the biological process by which sugars such as are converted into ethanol which can be used as fuel for internal combustion engines. Starch or sugar-based feedstocks can be used to produce ethanol or ethyl alcohol. Large fermenters are used to convert sugar and yeast into ethanol. After fermentation, the ethanol is separated from the other fluids in a distillation process. The anhydrous ethanol can be blended with gasoline and then shipped to gasoline terminals or retailers.

A problem with large industrial ethanol fermentation facilities is that the production requires large scale machinery that produces large batches of ethanol. The ethanol must then be blended with additives, transferred to delivery trucks and delivered to gas stations. These large ethanol production facilities also require a large number of specially trained technicians to operate and monitor the refinery processing and equipment as it operates 24 hours per day.

What is needed is a more convenient micro refinery system for producing ethanol in smaller batches using machinery that can be remotely and/or automatically operated and monitored to maintain optimum performance efficiency by an untrained individual.

### SUMMARY OF THE INVENTION

The present invention is directed towards a micro refinery system that produces ethanol from sugar or alcoholic beverages. The micro refinery is coupled to a computer network that allows the micro refineries to communicate with various computers. The micro refinery can also include many sensors that provide system operating data. The micro refinery can provide status information and perform automated operations such as projecting the required feedstock supplies based upon reserves or consumption rates. The system can monitor the feedstock reserves and estimate the required supplies needed to maintain the user's consumption of ethanol.

The micro refinery can also provide production information via electronic communications to any regulatory entity. The system can also indicate if there are any problems with any of the processing mechanisms. If the system requires any maintenance or repair, the micro refinery can transmit a signal indicating the need for required servicing. The system can transmit the information to the user computer so that the user can then request the servicing or in another embodiment, the system can automatically transmit the service request to the service provider.

When a micro refinery system is installed at the user's site the system can be coupled to a power supply, a water supply and a fluid drain. Because the system includes a fuel pump, it should be installed at a location easily accessible to vehicles. Set up of the micro refinery requires placement on a level surface and connecting it to a source of water, power and waste water disposal. The micro refinery operates automatically and the system operations are monitored and controlled by a user computer or a system management operator computer. The system can be monitored and controlled through a user interface and/or the data can be transmitted through a transmitter to a computer network such as the Internet. The operating information and system controls can then be transmitted through the network to a user's computer or a system management computer so that the operations can be monitored remotely.

When the system is installed at a user's location and the user turns the micro refinery on, the system may go through a start up process to check each system for proper operation. Once the system is ready to begin processing, feedstock which includes sugar, yeast and yeast nutrients, is mixed with water in the fermentation tank. The fermentation tank is a sealed unit that includes an agitator, temperature control mechanisms and load cells. A measuring system detects the quantities of the feedstock components placed in the fermentation tank and the volume of water mixed with the feedstock so that the fermentation process can begin. A control system manages the pumps, agitator, valves, fans, sensors and thermoelectric coolers that automatically maintain the proper chemical mixture and fermentation environment. In this process yeast consumes sugar and converts it to ethanol, carbon dioxide gas and heat. In an embodiment, the system can monitor the fermentation process by detecting the carbon dioxide emitted and detecting the chemical components of the batch. The insertion of the feedstock can be done manually or automatically through actuated valves that control the flow of materials into the tank.

The system can detect the initial weight of the batch ingredients by monitoring the output signals from the load cells. If the feedstock components are input sequentially and individually, the weight of each component is represented by the change in weight as each feedstock component is added. Based upon the detected feedstock weight, the corresponding proper weight/volume of water can be calculated. The quantity of water inserted into the tank can be detected by the load cells or a flow measurement mechanism. In an embodiment, the tank can also have a sensor that detects the volume of liquid in the fermentation tank. The liquid sensor can include a float connected to a variable resistor within the tank. When the tank is full, the resistor is set to its low resistance value. As the tank empties, the float slides along the resistor altering the resistance. The downward movement can increase the electrical resistance of the liquid sensor and the resistance may reach its highest value when the tank is empty. The system may be calibrated to calculate the volume of liquid based upon the detected electrical resistance.

The system may then mix the feedstock, sugar and water with an agitator. As the fermentation process takes place, the reaction of the yeast and sugar produces heat. The system can also control the temperature within the fermentation tank so that it stays within an optimum temperature range for fermentation. By monitoring the temperature, the heating system can determine if the fermentation tank needs to be heated, cooled or maintained. In an embodiment, the temperature control unit is a thermal plate that is coupled to the fermentation tank and is able to both heat and cool the interior volume by switching the polarity of the voltage applied to the plates. In the fermentation process, the feedstock and sugar are converted into ethanol and carbon dioxide. The carbon dioxide is vented from the fermentation tank and the weight of the materials in the tank decreases as the ethanol is produced. By detecting the change in weight of the materials over time, the system can identify the status and progress of the fermentation process.

In an embodiment, the system can check the mixture of fermentation ingredients to determine if the mixing ratios or recipes are proper and the batch is fermenting. During fermentation, samples of the batch liquid are periodically removed from the fermentation tank and placed in a testing area that contains one or more sensors. A tube coupled to a pump can be used to remove the sample from the batch and place the sample in the test area. The sensors can detect physical properties such as conductive, pH level, optical refraction or optical wavelength absorption and fluorescence. The sensor outputs are analyzed to determine the quantities of the chemical components in the fermentation tank. After the sample has been tested, the system can return the sample to the batch. In order to monitor the fermentation processing, the system can test samples of the batch continuously or periodically throughout the batch process. Thus, the sample removal system can continuously pump a portion of the batch into the test container or may periodically transport the test samples to the test container. In an embodiment, the batch can be tested directly in the fermentation tank without moving samples to a separate test chamber.

Once the sensors have taken measurements of the batch sample, the detected characteristics are compared to expected or optimum values which can be stored on a preprogrammed look up table of values or derived from an algorithm. By comparing the detected chemical components to the look up table or remote communication feedback the system can determine if the batch contains the optimum mix of batch ingredients. If there are substantial differences between the detected and expected values, the system can identify any deficient or excessive chemical ingredients. The system can then inform the user of the chemical ingredients that should be added to the tank to correct the batch. In other embodiments, the chemicals are stored internal and the micro refinery will automatically add materials to the fermentation tank to correct the chemical imbalance of the batch mixture.

In some cases the optimum mixture or ratio of ingredients can be based upon the ambient conditions. While the system can control the temperature of the tank, additional sensors measure ambient climate changes such as: temperature, humidity, barometric pressure, etc. When a change in the climate is detected, the system can consult a look up table or obtain online remote feedback for the proper ratio of ingredients. The system can then modify the mix of materials in the tank to the ideal recipe for the ambient conditions. The system will also turn on cooling or heating components to maintain proper climate within the tank.

After the fermentation processing is complete, the fermentation tank includes ethanol and water. The ethanol is separated from the water in a distillation system. Over a period of several days, it is slowly heated and pumped from the fermentation tank through a distillation column which creates a vapor of ethanol and water. Because the boiling points of these materials are different, the ethanol vapor will tend to rise to the top of the distillation tube while most of the water vapor condenses on the tube wall and does not exit the distillation tube.

Because the system can be mounted on an uneven surface and vertical alignment is necessary for proper distillation, the system includes a vertical alignment system for the distillation tube. In an embodiment, a gimbal is coupled to the upper half of the distillation tube. Because most of the weight is below the gimbal, the distillation tube will tend to rotate into vertical alignment. The system may only allow free rotation during an alignment process. After the alignment is performed, the system may lock the distillation tube in place to prevent rotational movement during the distillation process.

The distillation tube can be filled with material packing or horizontal perforated plates which are used to strip vaporized beer from the alcohol. Ideally, the vaporized beer and ethanol enter the bottom of the distillation tube and the beer vapor. Water and other heavier material are blocked by packing or plates. In contrast, the ethanol will tend to stay in vapor form and continue to travel up the distillation tube. This helps to separate the water and other contaminants from the ethanol vapor.

In an embodiment, multiple plates are vertically offset within the tube. However, in a preferred embodiment, the perforated plates that are angled diagonally are placed within the tube. The size of the holes in the perforated plates can be adjusted to optimize distillation performance. A potential problem occurs when the micro refinery has been running for a long time or temporarily stops production. The beer can remain on the packing or perforated plates within the condensation tube causing clogging of the perforations and/or packing. The entire condensation tube must then be cleaned before the system can operate at optimum efficiency. In order to reduce this clogging problem, the plates can be angled so that when water and beer vapor condenses on the plates, gravity will tend to draw the condensed liquid towards the lower edges of the plates. Because the beer liquids will tend to move away from the perforations and damage to the distillation tube is much less likely.

In an embodiment, the distillation tube is a multi-piece unit that can be disassembled into multiple pieces. If the plates or packing need to be replaced or repaired, the distillation tube can be disassembled and the interior surfaces can be accessed. In an embodiment, the distillation tube can have one or more couplings along the length. The couplings can be any suitable design. In a preferred embodiment, the couplings can include pins that engage hooks when sections of the distillation tube are rotated. Rotation in one direction will cause the sections to be coupled in axial alignment and rotation in an opposite direction will allow the sections to be released and separated. When the sections are coupled together, a locking mechanism can be used to prevent the sections from rotating.

Vapors exiting the distillation tube are passed through a separation membrane that separates the ethanol from the other fluids. The membrane can be damaged by the thermal shock of being exposed to hot vapors too quickly. In order to prevent damage to the membrane from the hot vapors, the system may include a pre-heating mechanism and a temperature sensor that detects the membrane temperature. The pre-heating mechanism can be a heater that gradually heats the membrane before it is exposed to the hot vapors. The preheating can be controlled by a controller that can adjust the rate of heat increase to prevent damage to the membrane. When the membrane temperature is similar to the temperature of the hot water and ethanol vapors, the system can open a valve to allow the water and ethanol vapors to flow through the membrane.

The membrane has small holes that allow the smaller water molecules to pass but not the larger ethanol molecules that stream through the exit port. A vacuum can be used to draw the water vapor through the membrane holes. After the water has been separated from the ethanol, a number of heat exchangers and thermal electric coolers convert the ethanol and water vapors back into liquids. The water is recycled and the fuel grade ethanol flows into a storage tank and is available for use. In an embodiment, the micro refinery stores the ethanol as well as gasoline. In an embodiment, the user controls the blending ratios of the ethanol to gasoline. When fuel is needed, the blended fuel is dispensed just like at a gas station through a hose and nozzle.

The micro refinery may also contain sensors that can detect system component performance and equipment failures. Various types of sensors can be included in the system. Pressure sensors are used to detect pump, column flow and motor pump fault conditions. Electrical and optical sensors can measure sugar, yeast, various nutrients, alcohol, water and pH balance contents. Temperature sensors measure internal column, heat exchangers, water storage level, ethanol storage level, thermo electric coolers, thermo electric heaters and ambient distillation closet and outer ambient climate. Accelerometers or strain gauge sensors measure fermentation tank weight. Voltage and current sensors measure column heating element, motors, pumps and power supply input and output voltages. Radio sensors detect Wi-Fi or data cellular networks. Through these sensors, the system operations can be monitored. The sensor data can be transmitted to the controller which can analyze, process and display the detected information from the sensors.

### BRIEF DESCRIPTION OF THE DRAWINGS

FIG. 1 is a side view of an embodiment of the micro refinery system;

FIG. 2 illustrates a thermoelectric mechanism;

FIG. 3 is a graph showing the change of the batch weight over time;

FIG. 4 illustrates a view of a gimbal mechanism;

FIG. 5 illustrates a top view of a distillation tube plate assembly;

FIG. 6 illustrates a side view of a distillation tube plate assembly;

FIG. 7 illustrates a distillation tube with a plurality of couplings;

FIGS. 8-10 illustrate an embodiment of a distillation tube coupling;

FIG. 11 illustrates a cross section of a porous membrane used to separate water and ethanol;

FIG. 12 illustrates the system controller and the connections to the sensors and control mechanisms of the micro refinery system;

FIG. 13 illustrates communication connections between the micro refineries and other system computers;

FIG. 14 illustrates a sensor for monitoring the fluorescence of the batch;

FIG. 15 illustrates an optical wavelength absorption sensor for monitoring the batch;

FIG. 16 illustrates an optical refraction sensor for monitoring the batch;

FIG. 17 illustrates a pH sensor for monitoring the batch;

FIG. 18 illustrates an oxygen sensor for monitoring the batch; and

FIG. 19 illustrates an electrical resistance sensor for monitoring the batch.

### DETAILED DESCRIPTION

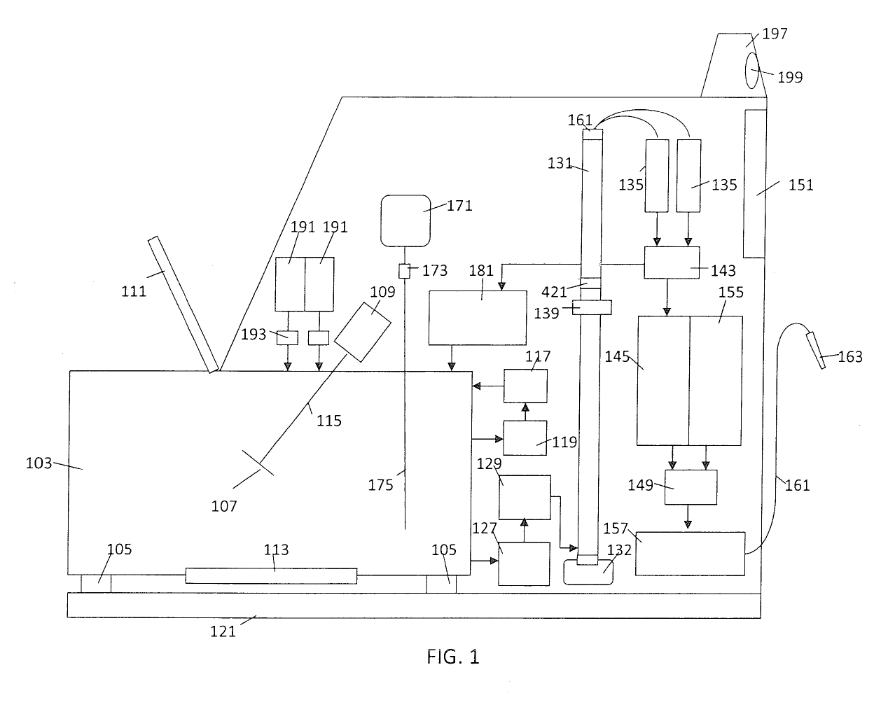

The components of the inventive micro refinery system 101 will be described with reference to FIG. 1. In an embodiment the fermentation tank 103 rests on one or more load cells 105 that detect the downward force and produce corresponding electrical output signals. The load cells 105 are coupled to a system controller 151 that monitors the weight of the tank 103 and all contents within the tank 103 throughout the ethanol conversion process. The load cell 105 output signals are proportional to the detected weight. In an embodiment, the system controller 151 can go through a calibration process which detects the weight of the empty tank 103 and stores the empty tank weight as an offset value. The offset value can then be subtracted from any detected weight so that the system controller 151 can detect the weight and quantity of materials that are inserted into the tank 103. The fermentation tank 103 calibration process may be repeated each time a batch of materials is processed.

The system controller 151 may provide a display and/or audio instructions which may indicate the sequence of materials and quantities to be inserted based upon the estimated quantity of ethanol to be produced. For example in an embodiment, a user may input the quantity of ethanol desired. The system then calculates the expected quantities of materials required to produce the desired quantity of ethanol and instructs the user to insert specific quantities of sugar and feedstock. To start the fermentation process, the lid 111 is opened and a specific ratio of sugar and feedstock are inserted into the tank 103.

In an embodiment, the sugar is the first material added to the fermentation tank 103. The weight of the sugar is detected by the system controller 151 and the corresponding volume of water is determined. After the sugar has been added, the system controller 151 can instruct the user to insert the feedstock. The system controller 151 can detect the weight of feedstock and provide instructions and information regarding the quantity of feedstock to add to the fermentation tank. The system controller 151 can detect the weight of the materials being inserted and may provide instructions to the user such as: add more, slow the rate of insertion in preparation to stop and stop. The system controller 151 may have a visual display that indicates the volume of materials added to the tank so the user knows when to stop adding materials to produce the desired volume of ethanol. The system controller 151 may also provide feedback if errors are made. For example, if the system controller 151 detects that too much sugar was added, the system may compensate for this error by increasing the quantity of feedstock to be added to the fermentation tank 103 for the extra sugar.

In another embodiment, the sugar, yeast and other feedstock components such as: phosphorus, sulfur, potassium, magnesium, minerals, amino acids and vitamins can be stored in containers 191 that are coupled to the fermentation tank 103 and the control system 151 can control valves 193 coupled to the containers. Thus, the control system 151 can add the required materials into the fermentation tank 103 so that the insertion of the sugar, yeast and other components is automated. The system may also allow for the large initial quantity of materials to be manually inserted into the fermentation tank and then add additional materials stored in the containers to adjust the batch as necessary. When the proper volume and ratio of feedstock and sugar have been inserted into the fermentation tank 103, the lid 111 is closed. The lid 111 may have a locking mechanism to prevent the addition of any other materials to the tank 103 until after processing is completed.

As discussed, the system controller 151 detects the quantity of sugar in the fermentation tank 103 and calculates the corresponding volume of water for the fermentation process. The system can automatically add the volume of water required for fermentation processing to the tank 103. The proper volume of water can be detected based upon a metered flow of water from a water storage tank 181. Alternatively, the system controller 151 can detect the weight of the water and calculate the volume of water added based upon the known volumetric weight. The system controller 151 is coupled to a valve between the water tank 181 and the fermentation tank 103. The system controller 151 can open the valve to cause water to flow into the tank 103 and when the proper volumetric weight change is detected, the system controller 151 can close the valve. In other embodiments, the water can be added to the fermentation tank 103 manually and the system will indicate when the proper quantity of water has been added.

With the proper mixture of water, feedstock and sugar in the fermentation tank 103 the system can mix the batch ingredients by rotating the agitator 107 to mix the materials. In an embodiment, a motor 109 is used to rotate shaft 115 coupled to an agitating element 107. The agitating element 107 can be an elongated angled mixing blade that circulates liquids in the tank 103 when rotated. The mixing is required to cause the yeast in the feedstock to come in contact with the sugar and nutrients required for fermentation. While a single agitator 107 is illustrated, in other embodiments multiple agitators can be used to mix the materials and prevent clumping of the sugar and feedstock in the corners of the tank 103.

In an embodiment, the control system 151 may detect the proper mixing of the batch materials by the rotational resistance of the agitator 107 or viscosity. A low resistance or viscosity indicates that the agitator 107 is only in contact with water while a higher resistance may indicate that the agitator 107 has contacted a clump of sugar or feedstock. The system can be configured to move the agitator 107 and the shaft 115 within the fermentation tank 103 to completely mix the batch materials. During the mixing process, the rotational resistance is an indication of the status of the mixing. The materials may be properly mixed when the rotational resistance is steady and corresponds to a proper resistance range for the mixture. Once the proper mixed viscosity is detected, the materials are properly mixed and the rotation of the agitator 107 can be stopped or run periodically during the fermentation process.

During the fermentation process, the yeast absorbs the sugar when diluted in water. This reaction produces 50% ethanol and 50% CO₂ by the end of the fermentation process. The chemical equation below summarizes the conversion:

C₆H₁₂O₆(Glucose)→2CH₃CH₂OH(Ethanol)+2CO₂+heat

In other embodiments, the micro refinery is able to process cellulosic materials to produce ethanol. Cellulosic ethanol is made from plant waste such as wood chips, corn cobs and stalks, wheat straw and sugarcane stalks, stems and leaves or municipal solid plant waste. An advantage for a cellulosic fuel production is that the micro refineries can be configured to process the regional crop plant material, reducing delivery costs. For example, the micro refineries located in the Midwest can be configured to process: wheat straw and corn residue. In the Southern United States the micro refinery can process sugarcane. In the Pacific Northwest and Southeast, wood can be converted into Ethanol.

Corn is easily processed because corn has starches that enzymes can easily break down into sugars and yeast ferments the sugars to produce ethanol. In contrast, cellulosic stalks and leaves contain carbohydrates that are tougher to break down and unravel because they are tightly bound with other compounds. Thus, special processing is required make ethanol from cellulosic farm waste. More specifically, special enzymes are needed in the fermentation tank to break down the carbohydrates. In addition to the special enzymes, the farm waste processing requires genetically engineered bacteria to ferment the farm waste sugars into ethanol.

Another problem with farm waste is that it can be mixed with earth matter such as rocks, clay and gravel that can damage the micro refinery components. In order to prevent damage, the cellulosic materials can be ground with a grinder to more finely chop the materials before processing. The cellulose materials are also separated into glucose and non-glucose sugars using a machine that applies heat, pressure and acid to the cellulosic materials. The heat and pressure produce a sugar and fiber slurry mixture. The non-glucose sugars are washed from the fibers and the glucose based fibers are processed with enzymes to break down and separate the sugars from the fibers. The separated sugars are then fermented with special bacteria microbes into a beer containing ethanol, water and other residue. After fermentation, the micro refinery vaporizes the beer so that the ethanol vapors rise up through a distillation tube to separate the ethanol from water. The vapor from the distillation tube is processed by a porous filter that is used to separate the ethanol vapor from any remaining water vapor as described above.

In another embodiment a different process is used to separate the glucose and non-glucose sugars. The mixture of glucose and non-glucose sugars can be separated, by mixing cellulosic materials with a solution of about 25-90% acid by weight. The acid at least partially breaks down the cellulosic materials and converts the materials into a gel that includes solid material and a liquid portion. The gel is then diluted from about 20% to about 30% by weight and heating the gel, thereby at least partially hydrolyzing the cellulose contained in the materials. The liquid portion can then be separated from the solid material, thereby obtaining a mixed liquid containing sugars and acids. The sugars are then separated from the acids in the mixed liquid by resin separation to produce a mixed sugar liquid containing a total of 15% or more sugar by weight and an acid content of less then 3% by weight.

The method of obtaining the mixed sugar further comprises mixing the separated solid material with a solution of about 25-90% sulfuric acid by weight, thereby further breaks down the solid material to form a second gel that includes a second solid material and a second liquid portion. The second gel liquid is diluted to an acid concentration of from about 20% to about 30% by weight. The diluted second gel liquid is then heated to a temperature between about 80° to 100° C., thereby further hydrolyzing the cellulose remaining in the second gel. The second liquid portion is separated from the second solid material to obtain a second liquid containing sugars and acid. The first and second liquids can be combined to form a mixed liquid. The glucose separation process is described in more detail in U.S. patent application Ser. No. 10/485,285 filed on Jan. 26, 2004, which is hereby incorporated by reference. The described process for producing ethanol from cellulosic materials has many benefits. Tree remains, lawn clippings and other plant debris are normally disposed of in landfill. By using these materials to produce ethanol, the land fill created is significantly reduced, the micro refinery has a substantially free source of feedstock and less greenhouse gases are produced.

A requirement of fermentation is proper temperature control to keep the ingredients within a proper fermentation temperature range. If the yeast temperature is too cold the yeast can become dormant and fermentation is slowed and if the temperature is too high the yeast can be killed. There are various types of yeast, some of which have a high temperature tolerance. The internal temperature of the fermentation tank 103 should be between about 60 and 90 degrees Fahrenheit to preserve yeast culture life. In order to increase the speed of fermentation, the temperature may be maintained at the higher end of the yeast tolerance temperature range.

In an embodiment, the system 101 also includes a thermoelectric mechanism 113 that can be coupled to the fermentation tank 103. The thermoelectric mechanism 113 is powered by a DC electrical power supply and maintains the optimum processing temperature within the tank 103. In order to provide uniform temperature control, a plurality of thermoelectric mechanisms 113 can be attached to various sections of the tank 103. In an embodiment, the system controller 151 is coupled to the thermoelectric mechanism 113 and a temperature transducer is mounted within the fermentation tank 103. The system controller 151 receives a signal corresponding to the internal tank temperature from the temperature transducer and determines if the fermentation tank 103 is within the proper temperature range or if the batch needs to be heated or cooled. As discussed above, the fermentation process produces heat, so in some cases heating or cooling of the tank 103 may not be required. If the system detects that the fermentation tank 103 is too cold, the system controller 151 applies direct current electrical power to the thermoelectric mechanism 113 in the heating mode of operation. If the temperature of the fermentation tank 103 is too hot, the thermoelectric mechanisms 113 can be switch to a cooling mode to reduce the temperature of the tank 103 by reversing the polarity of the electrical power to the thermoelectric mechanism 113. The system controller 151 can also turn the power to the thermoelectric mechanism 113 off when the fermentation tank 103 temperature is within the proper or optimum temperature range for fermentation. The optimum temperature can depend upon the specific type of yeast being fermented but is typically between about 25° C. to 30° C.

In another embodiment, the system may utilize a pump 119 that pumps the batch through a thermoelectric radiator 117 that is separate from the fermentation tank and then returns the batch to the fermentation tank. If the system controller 151 detects that the batch is too cold, the pump 119 is actuated to pump the batch through the thermoelectric radiator 117 which is controlled by the controller 151 to heat the batch. Alternatively, if the system controller 151 detects that the batch is too hot, the pump 119 is actuated to pump the batch through the thermoelectric radiator 117 which is controlled by the controller 151 to cool the batch. The outlet of the thermoelectric radiator 117 can be coupled to the fermentation tank 103 so that all thermally processed batch materials are returned to the fermentation tank 103.

In an embodiment, the system can be used in a wide variety of environments and has the ability to produce ethanol in a wide range of ambient conditions. This requires the cooling of the fermentation tank in hot regions and seasons and heating of the fermentation tank 103 in cold areas and seasons. A larger number of thermoelectric mechanisms 113 can be used in systems located in more extreme ambient temperatures. In an embodiment, the user can simply purchase and install additional thermoelectric mechanisms 113 to compensate for the hotter or colder temperatures. It is also possible to reduce the effects of extreme ambient temperatures by placing the micro refinery system within a protective enclosure and adding insulation to the micro refinery systems.

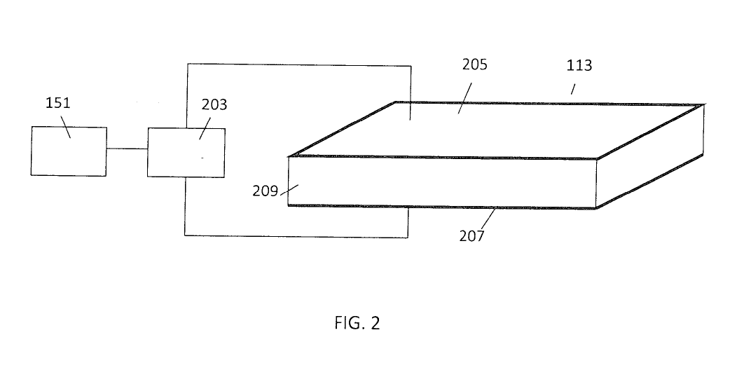

With reference to FIG. 2, in an embodiment the thermoelectric heating and cooling mechanism 113 can have two metal plates 205 and 207 that surround a ceramic core 209. When electrically charged, the plates 205 and 207 oscillate creating a cool side 205 and hot side 207. In an embodiment, the two plates 205 and 207 always maintain a 69 degree temperature difference between the two sides of 205 and 207. The voltage is applied to the plates 205 and 207 by a DC power source 203 which is controlled by the system controller 151. The heating or cooling output of the thermoelectric mechanism 113 can be controlled by reversing the polarity of the applied electrical power from the power source 203. Since thermoelectric mechanisms 113 are small in size, about 2 to 4 inches in diameter, the thermoelectric mechanisms 113 are ideal for small cooling and heating applications such as the batch materials in the fermentation tank.

The thermoelectric mechanisms 113 can be mounted on the fermentation tank 103 walls or, as discussed above with reference to FIG. 1, the thermoelectric mechanisms can be configured as a thermoelectric radiator 117. The fermentation liquid can be pumped through a thermoelectric radiator 117 to provide heating and cooling. Thus, the thermoelectric heating and cooling mechanism 113 and thermoelectric radiator 117 can cool the batch fermentation tank or heat the batch through the system controller 151 by reversing the DC polarity applied to the thermoelectric mechanisms 113 and thermoelectric radiator 117.

In a preferred embodiment, the fermentation tank 103 holds about 200 gallons of liquid. The thermoelectric mechanisms 113 are practical for small fermentation batches in this liquid volume range, but lack enough thermal energy to perform thermal control of larger commercial fermentation processing. For these reasons, the thermoelectric mechanisms can be used with the inventive system to control the temperature of about 200 gallons of liquid but are not suitable for temperature control of a larger 1,000+ gallon commercial fermentation processing tank.

A problem with the fermentation process is that it is not always a predictable process. The time required to complete the fermentation process will vary depending upon the purity of the sugar, and yeast, as well as the batch temperature. One way to monitor the fermentation progress is by monitoring the change in weight of the fermenting liquid. During fermentation, the sugar is converted into ethanol and CO₂ which is vented out of the fermentation tank 103. Thus, the venting of the CO₂ results in a weight reduction of the batch. In an embodiment, the force sensors 105 are used to periodically or continuously check the weight of the batch during the fermentation process. As CO₂ is vented from the fermentation tank 103, the batch gets lighter. The system can monitor the progress of batch fermentation by monitoring changes in the weight of the batch. An initial weight of the batch can be determined and stored in memory. Changes in the batch weight are caused by the conversion of sugar into CO₂ which is vented from the fermentation tank 103. The system controller 151 can determine that the fermentation process is complete when the weight of the batch is reduced by a known percentage. Alternatively, the system controller 151 can determine that the fermentation process is complete when the rate of weight reduction slows or stops. A CO₂ sensor can also be coupled to the fermentation tank. Since the CO₂ is vented, a low level of CO₂ in the tank 103 would indicate that less CO₂ is being produced by the batch.

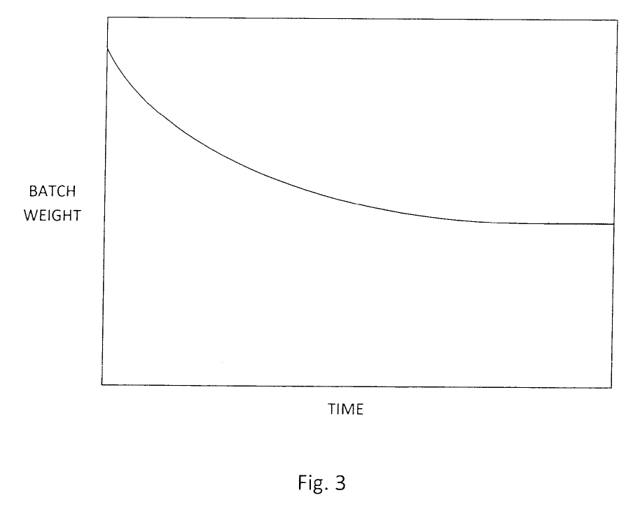

As discussed above, the force sensors 105 can be used for detecting an initial start weight of the sugar, feedstock and water loaded into the tank 103 at the beginning of the fermentation process. The weight can then be detected periodically by sampling the force sensors 105 at time intervals. By monitoring the weight of the batch over time, the rate of weight change over time can be used to determine the stage of the batch in the fermentation process. For example with reference to FIG. 3, a graphical representation of the weight of the batch over time is illustrated. At the beginning of the process, the weight of the batch drops fairly quickly. As the conversion of the sugar to ethanol progresses, the rate at which the weight decreases slows. Eventually, the weight change becomes very low indicating that the fermentation process is complete.

In addition to detecting the weight of the batch, the system can also perform chemical detection of the batch ingredients. In an embodiment, the micro refinery includes a batch testing mechanism 171 shown in FIG. 1, which can detect the chemical components of the batch and may include an optical, electrical, chemical or any other type of chemical sensor. A delivery mechanism may include a tube 175 that is coupled to a pump 173 to deliver samples of the batch to the testing mechanism 171. The testing mechanism 171 can be coupled to the controller 151 and can be used to check the chemical balance of the batch during the fermentation process. The detected quantity or ratio of batch components from the test mechanism 171 is compared to an optimum value which can be stored on a look up table or provided by another source. The optimum ratio of the batch components can change during fermentation. If there is a significant difference between the measured and optimum values, the controller 151 can transmit a signal indicating the problem and/or the controller 151 may automatically add chemical components to the fermentation tank 103 to rebalance the batch. By continuously testing and adjusting the batch throughout the fermentation process, the ethanol production from the batch can be maximized. More specific examples and descriptions of the sensors used in the chemical testing mechanism are described later.

Although the fermentation tank 103 has been described above for fermenting sugar and feedstock, the inventive system also has the ability to process different materials and can extract ethanol from recycled alcoholic beverages such as beer, wine and other alcohol products. The user can select the function of the micro refinery system as either a sugar fermentation tank or a processor of discarded alcohol. In the sugar fermentation mode, the micro refinery system ferments the sugar to create alcohol as described above. In the alcohol recycling mode, the alcoholic products also go into the fermentation tank prior to being processed by a distillation system for conversion into ethanol. The multi-function design provides a market advantage for recycling either sugar or discarded alcohol commonly found at bar restaurants or wineries.

After or during the fermentation of the sugar, it is possible to add the alcoholic liquids to the fermentation tank. The processor can indicate when alcoholic beverages can be added. In an embodiment, the controller can actuate a locking mechanism coupled to the lid 111 to allow or prevent the user from adding materials to the fermentation tank 103. Because the reaction of the yeast has converted much of the liquid into carbon dioxide, the volume of liquids in the fermentation tank 103 will decrease after fermentation is complete which allows room for recycling the alcoholic beverages. The micro refinery will then separate the ethanol from the batch as well as the alcohol from the discarded beverages and the other liquid components.

The ethanol is separated from the water and other liquids by processing the fluids through a distillation system. In an embodiment, the distillation system of the present invention includes a pump 127, a heater 128, a distillation tube 131 and a gimbaled mechanism 139 that is used to position the distillation tube 131 in a vertical orientation. The vertical orientation can be maintained by a gyroscope 132 mounted to the distillation tube 131. The gyroscope 132 includes a rotor that can be aligned with the vertical axis of the distillation tube and a motor that rotates the rotor. The rotation of the rotor stabilizes the gyroscope 132 and distillation tube from any rotational movement. The control system 151 controls the pump 127 to pump the liquids in the fermentation tank 103 through the heater 129 to cause the water and ethanol to boil and vaporize. The vaporized liquid is directed to the bottom of the distillation tube 131. As the vapors travel higher through the distillation tube 131, the ethanol molecules separate from the water molecules and exit the upper part of the column. If water and other non-ethanol liquids vaporize, these vapors will tend to be condensed on the sides of the distillation tube as they cool in the distillation tube 131. The condensed liquids may then adhere or drip down the inner walls of the distillation tube 131 rather than exiting the top of the tube 131. The distillation system may also include one or more temperature sensors which monitor the vapor temperature and control the heater 128 to produce vapor at an optimum separation temperature. Excessive heat will cause a faster vapor velocity resulting in more water exiting the distillation tube 131, while a low temperature vapor temperature will result in a low flow of ethanol from the distillation tube 131.

The distillation process requires that the distillation tube 131 be in a perfect vertical alignment. The vapors slowly rise vertically straight up and the flow path is preferably undisturbed by sidewalls as the vapors travel up through the center of the distillation tube 131 and out from the top. If the distillation tube 131 is out of alignment, the rising vapors will run into the side of the tube 131 resulting in condensation of ethanol vapors and reducing the efficiency of the distillation system. Similarly, water vapor rising on the side wall tilted away from vertical may not condense on the sidewalls reducing the separation of the water and ethanol. Thus, perfect vertical alignment is necessary for the high efficiency distillation.

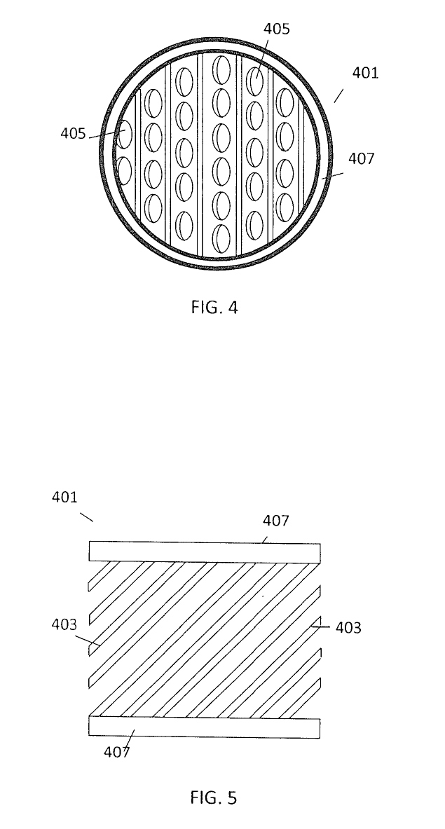

With reference to FIG. 4, the gimbaled mechanism 139 supports the distillation tube 131 and allows the tube 131 to naturally rotate into vertical alignment. In an embodiment, the gimbaled mechanism 139 includes an inner pivot 305 that couples the distillation tube 131 to a ring 309 that surrounds the tube 131 and an outer pivot 307 that couples the ring 309 to a fixed support 311 that is coupled to the frame. The pivots 307 and 309 may include low friction bearings or bushings which allow the pivots 307 and 309 to rotate with very low friction. The gimbaled mechanism 139 is mounted above the center of gravity of the distillation tube 131 so that the weight of the distillation tube 131 below the gimbaled mechanism 139 will cause the tube 131 to automatically self align in a vertical orientation. If the fixed support 311 is rigidly mounted to the frame, the distillation tube 131 automatically rotate to vertical alignment.

In an embodiment, a gyroscope 132 shown in FIG. 1 is mounted to the bottom of the distillation tube 131. The gyroscope 132 includes a rotor and a motor that rotates the rotor. Because the weight of the gyroscope 132 is supported by the distillation tube 131, the center of gravity of the gyroscope 132 can be aligned with the vertical center axis of the distillation tube 131 so the weight will not cause misalignment. The rotational axis of the rotor can be aligned with the vertical axis of the distillation tube and while the rotor is rotating the gyroscope 132 and distillation tube 131 are stabilizes so that any angular motion of the micro refinery will not alter the vertical alignment of the distillation tube. In an embodiment, a distillation tube 131 is vertically aligned before the gyroscope is turned on and the rotor starts spinning.

The distillation tube 131 can be fragile and in some cases it may be desirable to lock the distillation tube 131 in place to prevent movement. In an embodiment, the vertical alignment system includes a locking mechanism that prevents the distillation tube from rotating. In an embodiment, the system can detect ambient conditions through sensors such as wind meters and/or accelerometers coupled to the housing. If the wind speed is very high, the system may move which will cause the distillation tube to move out of vertical alignment. Rather than risking damage to the distillation tube, the system may have a “safe” mode that can be actuated when predetermined wind speed or acceleration movement is detected. For example, the micro refinery may go into a safe mode with the distillation tube and other fragile system components locked in a safe position, when the detected winds are greater than 40 MPH are detected or an earthquake greater than 5.0 is detected. The system may also receive weather warnings for its geographic location from an outside source such as the internet weather information services and respond to storm warnings by scheduling safe mode times. The controller may also shut off power and/or provide surge protection to prevent damage to the electrical components due to power surges or power outages.

In an embodiment, the distillation tube can be filled with material packing or horizontal perforated plates which are used to strip vaporized beer from the alcohol. Ideally, the vaporized beer and ethanol enter the bottom of the distillation tube and the combined vapor travels up the tube. Water and other heavier material are blocked by packing or plates. In contrast, the ethanol will tend to stay in vapor form and continue to travel up the distillation tube. This helps to separate the water and other contaminants from the ethanol vapor. The plates can be horizontally oriented within the tube and multiple plates can be positioned along the length of the distillation tube. A potential problem occurs when the micro refinery temporarily stops production. The water will condense or evaporate and the beer can remain on the packing or perforated plates causing clogging of the perforations or packing when the system is used again. The entire condensation tube may need to be cleaned before the system can be used again.

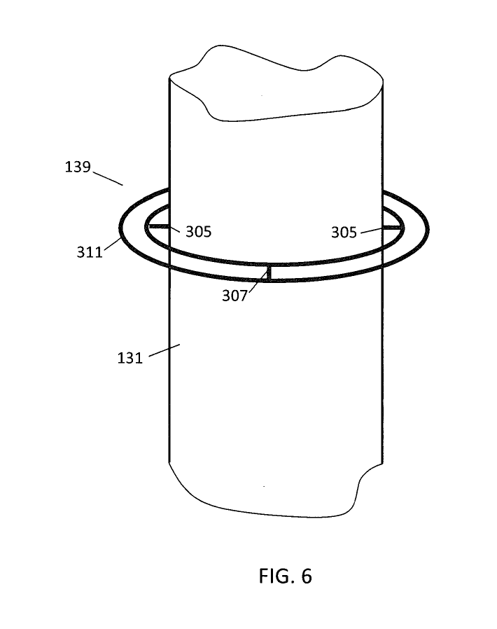

In order to reduce this problem, the perforated plates 403 can be mounted within the distillation tube 131 at an angle. FIG. 5 illustrates a top view of the plate assembly 401 and shows some of the holes 405 formed in the angled plates 403. FIG. 6 illustrates a side view of the assembly 401 and illustrates a possible angle of the plates 403. When water and beer vapor condenses on the plates 403, gravity will tend to draw these liquids towards the lower edges of the plates 403. Because the beer liquids will tend move away from the holes 405 towards the lower edge of the plate 403, damage to the distillation tube and plates 403 due to dried contaminants is much less likely to occur. When installed in the distillation tube, the rings 407 may fit closely within the inner diameter of the distillation tube. The assembly may also have upper and lower rings 407 that hold the components together as a single piece unit that can be easily installed or removed for repair or maintenance. The assembly 401 can be made of any suitable material including metals such as stainless steel and plastics. The assembly 401 may also be coated or painted with a protective finish that will prevent rust and oxidation. While the plates 403 are illustrated as planar structures in other embodiments, they can also be non-planar. For example, the plates can have an in inverted “V” shape so that condensed liquids will tend to flow to the lower opposite lower sides of the plate.

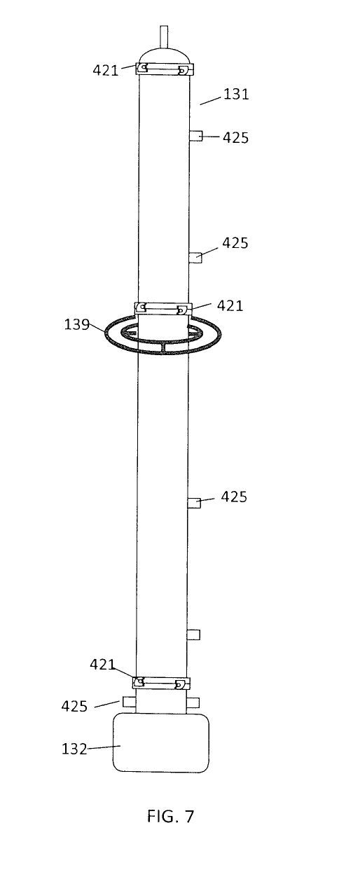

With reference to FIG. 7, a side view of an embodiment of the distillation tube 131 and associated hardware components are illustrated. In an embodiment, the distillation tube 131 is a multi-piece unit that can be disassembled into multiple pieces. In this example, there are three couplings 421 that are used to secure different sections of the distillation tube 131 to each other. However, it is contemplated that any number of distillation tube sections and couplings 421 can be used. The couplings 421 are releasable to allow the distillation tube 131 to be disassembled. This is particularly useful if the plates or packing need to be replaced or repaired or the interior surfaces need to be accessed for cleaning. This can reduce the time required from several hours to a few minutes. The multiple piece configuration also allows the distillation tube 131 to be more easily transported for field servicing and repair since a defective portion can be replaced if necessary rather than the entire tube 131. In order to further simplify the removal from the system, the distillation tube can have quick disconnect fittings 425 secured to the outer diameter of the distillation tube 131. The fittings 425 can be used for temperature and pressure sensors as well as the fluid inlet and outlet ports.

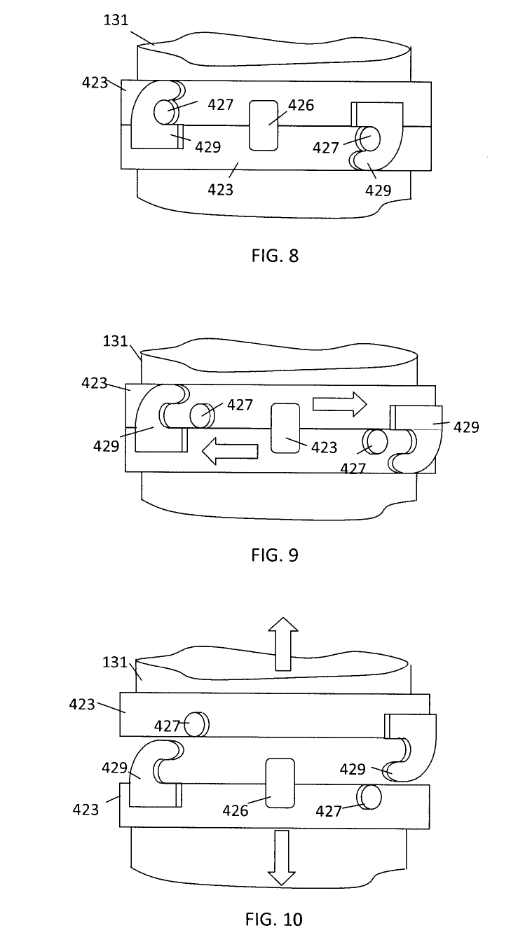

Details of an embodiment of a coupling 421 are illustrated in FIGS. 8-10. FIG. 8 shows the coupling 421 in the coupled state. The coupling 421 includes a plurality of pins 427 and hooks 429 that are mounted on rings 423 that are coupled to the outer diameter of the distillation tube 131 and provide structural support. The pins 427 and hooks 429 are distributed around the circumference of the rings 423. By rotating the sections of the distillation tube 131 the hooks 429 are secured to the pins 427 and the sections of the distillation tube are secured together. In an embodiment, a locking mechanism 426 is used to prevent the rings 423 from rotating so that the portions of the distillation tube 131 cannot be separated.

With reference to FIG. 9, when the distillation tube 131 needs to be disassembled, the sections are rotated so that the pins 427 disengage from the hooks 429. As shown in FIG. 10, with the pins 427 separated from the hooks 429, the sections of the distillation tube 131 can be separated from each other. In other embodiments, various other types of coupling mechanisms can be used to secure sections of the distillation tube together.

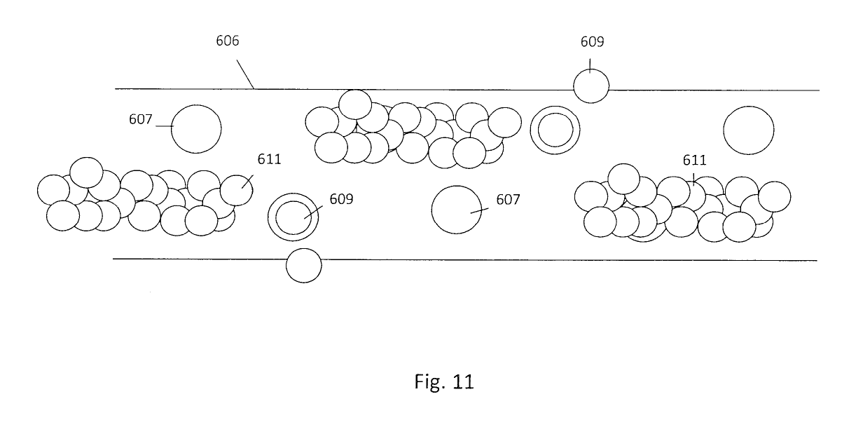

During the normal operation of the micro refinery, the hot ethanol and water vapors exit the distillation tube 131 and travel through a membrane system 135 which separates water molecules from the ethanol molecules. The membrane system 135 includes a porous separation membrane that can be made of ceramic, glass or very course materials. With reference to FIG. 11, the membrane 606 has many small holes 607 which allow water molecules 609 to pass through, but are too small for the ethanol molecules 611 to pass through. The higher pressure of the vapor causes the smaller water molecules 609 to flow through the holes 607. By passing the vapor through the membrane 606, the water molecules 609 are separated and substantially pure ethanol molecules 609 exits through the membrane system. Thus, the holes 607 are larger than the diameter of water vapor and smaller than the diameter of ethanol vapor. In an embodiment the holes 607 may be between about 2 and 40 angstroms in diameter.

A potential problem with the porous membrane system is that the membrane materials can be susceptible to this thermal damage. In particular, “thermal damage” of the membrane can occur if the temperature of the ethanol vapor is substantially hotter than the membrane. For example, the membrane may be at ambient temperature and then immediately exposed to hot ethanol vapor resulting in damage. To prevent thermal damage of the membrane a micro controlled warming system is used to pre-heat the membrane to ensure the membrane temperature is suitable for processing the hot vapor. In an embodiment, the temperature of the membrane is detected by a thermocouple attached to the membrane system. As the control system directs the flow of fluids out of the fermentation tank through to the heater and distillation tube, it detects the temperature of the membrane before the hot vapors are directed to the distillation tube. With reference to FIG. 1, if the membrane is cold, the system controller 151 can activate a heating element and monitor the membrane temperature. As the membrane temperature increases, the control system may have a thermostatic setting to prevent over heating of the membrane by the heater. When the membrane temperature is pre-heated to a safe temperature, the system controller 151 can allow hot vapors to flow through the distillation tube 131 to the membrane. Once the hot vapors are flowing through the membrane, the vapors will heat the membrane and power to the heating element can be removed. In order to assist with the ethanol and water separation process, the water vapor can be drawn through the porous membrane with a vacuum 143.

In an embodiment, the membrane system 135 can have a back up membrane 135. If one membrane system 135 is damaged, the controller will detect the failure and the controller 151 can actuate a valve 136 to divert the water and ethanol vapors from the distillation tube 131 to the back up membrane system 135. The controller 151 can transmit a signal indicating that the membrane 135 is damaged through the transceiver 197 to an operator or maintenance group. The damaged membrane system 135 can then be replaced while the water and ethanol vapors are separated by the backup membrane system 135.

After passing through the membrane system 135 and vacuum 143, the water can condense and flow into the water storage tank 181 before being used again in the fermentation tank 131. The separated ethanol exits the membrane system 135 and then flows through a thermoelectric cooler 166 which causes the ethanol to condense into a liquid. The liquid ethanol then flows into a storage tank 145 where it is stored before being mixed with gasoline. An ultrasonic or other liquid sensor coupled to the storage tank 145 can detect the liquid ethanol level within the storage tank 145 and provide this ethanol production information to the system controller 151. In an embodiment, the system controller 151 can detect when the ethanol storage tank 145 is full and stop the distillation process until there is available space in the storage tank 145.

In an embodiment, the inventive micro refinery can mix the ethanol stored in the ethanol storage tank 145 with gasoline that is stored in a gasoline storage tank 155 in any ratio set by the user through the system controller 151. The control system includes a user interface which allows the user to select the desired fuel blend ratio. The system may include a lock that prevents the fuel mixture setting to exceed the maximum or minimum allowable ethanol percentage for the vehicle. Once the fuel mixture has been selected, the user can use the micro refinery functions like a normal gasoline pump. The user removes the nozzle 163 from a cradle on the micro refinery 101 and places it in the tank filler of the vehicle. A lever coupled to the nozzle 163 is actuated to start the pumps 149 which cause the fuel to flow from the tanks 145 and 155 through the hose reel 157, the hose 161 and nozzle 163 to the tank filler of the vehicle. The system will run the ethanol and gasoline pumps 149 at different flow rates to produce the specified fuel ratio. The nozzle 163 will detect when the vehicle tank is full and automatically stop the flow of fuel through the nozzle 163. When the vehicle tank is full, the user places the nozzle 163 back in the cradle and replaces the cap on the fuel filler to end the filling process. With the ethanol tank 145 at least partially drained, the system can begin to produce more ethanol.

The mix ratio of ethanol and gasoline or other fuels can depend upon the type of vehicle being fueled. The use of pure ethanol in internal combustion engines is only possible if the engine is designed or modified for that purpose. However, ethanol can be mixed with gasoline in various ratios for use in unmodified automobile engines. In the United States, normal cars designed to run on gasoline may only be able to use a blended fuel containing up to 15% ethanol. In contrast, U.S. flexible fuel vehicles can use blends that have less than 20% ethanol or up to 85%. The ethanol fuel blend is typically indicated by the letter “E” followed by the percentage of ethanol. For example, typical ethanol fuel names include: E5, E7, E10, E15, E20, E85, E95 and E100, where E5 is 5% ethanol and 95% gasoline, etc.

After the processing performed by each of the micro refinery systems is complete, the micro refinery systems may also be cleaned. In an embodiment, the micro refinery includes cleaning mechanisms that can spray the fermentation tank with pressurized soap and water which will remove particulates from the tanks and other components. The system can then rinse the system components to remove the soap and other residue. In an embodiment a drain valve is opened to allow the waste liquids from the fermentation tank and the distillation system to drain from the system through a drain hose. The system may include an automated cleaning system that utilizes valves coupled between a water supply and a spray nozzle that emits high pressure water and is actuated by the system controller. The spray can be directed towards the fermentation chamber walls to remote deposited materials. As the volatile materials have been removed from the interior surfaces of the micro refinery, a drain valve is opened and the waste materials can be poured down into public drainage systems.

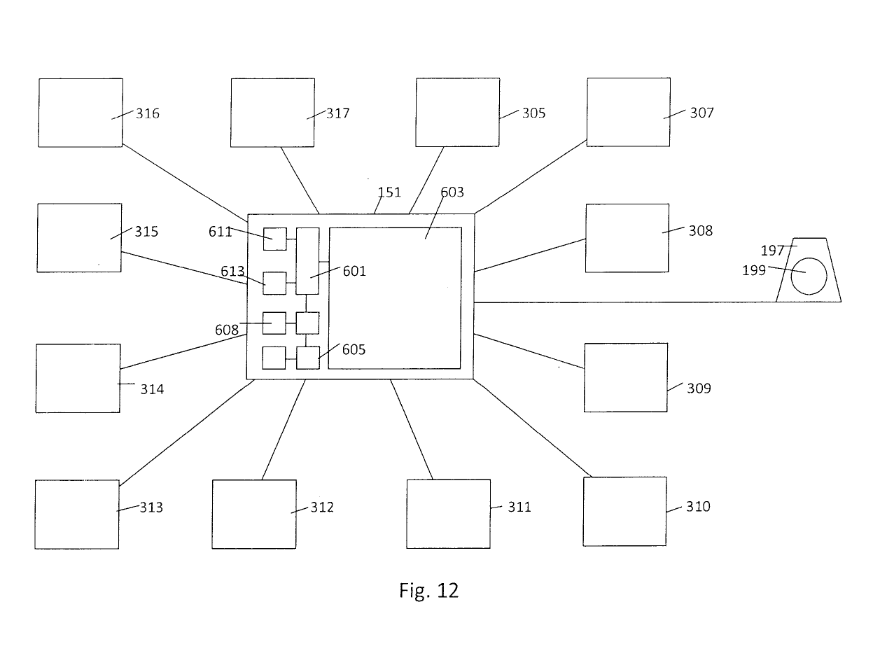

Because the micro refinery is a complex mechanism, sensors and controls are used to automate the operation and optimize the ethanol production performance. The micro refinery can include various sensors that monitor the operating conditions of the processing systems including: the fermentation tank, the load cell weight detection system, the temperature control system, the mixing agitator for the fermentation tank, the distillation system, the membrane separation system, the storage tank and a blending and pumping system. All of these systems include sensors that are coupled to the controller. With reference to FIG. 12, a simplified block diagram of the system controller 151 is illustrated. The controller 151 includes a central processing unit (CPU) 601, a visual display 603 which may also be an input device, user input mechanisms 609 such as buttons, key pads, etc. In order for the CPU 601 to communicate with analog devices such as motors, sensors, analog to digital converters 611 and digital to analog converters 613 are required. The analog to digital converters 611 are used to convert analog data signals into digital signals that can be interpreted by the CPU 601 and the digital to analog converters 613 are used to convert the digital control signals from the CPU 601 to the analog devices. The CPU 601 can also be coupled to an electronic memory 605. In an embodiment, the controller 151 is coupled to various sensors 305-313 that detect the operation of the micro refinery and system components and controllers 314-317 that control the operation of the micro refinery.

In an embodiment, the controller 151 can interpret the detected sensor 205 information which can emit an audio or visual status indicator. The controller 151 can also be coupled to a light 199 which provides an indication of the status of the micro refinery. The color of the light may indicate the system status. A green light may indicate that everything is OK, a yellow light may indicate that the system needs attention and a red light may indicate that the system has been shut down. As shown in FIG. 1, the light 199 can be mounted on an upper surface of the micro refinery 101 for optimum visibility. If the micro refinery 101 is located away from others, the status indicator light 199 can be mounted to a pole so that the status indicator can be easily seen from a distance by a user. In other embodiments, an audible alarm may be sounded in emergency situations when the system needs attention or the system has been shut down.

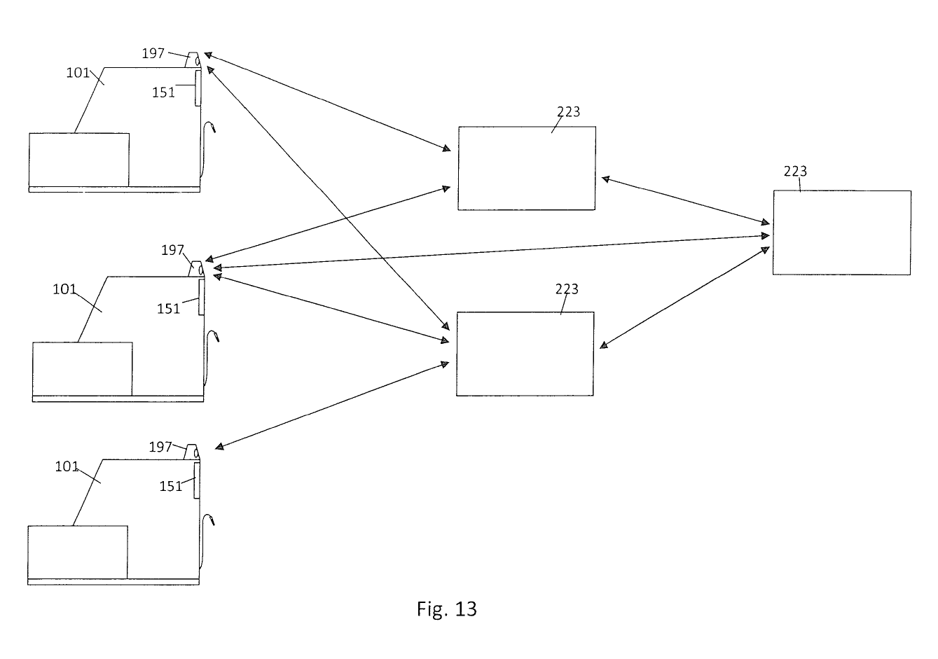

FIG. 13 is a diagram illustrating the communication connections between the micro refineries 101 and other system components. The controller 151 may also coupled to a transceiver 197 that can transmit and receive information through wired or wireless communications. For example, the micro refinery may communicate with a computer network which allows a computer to monitor the operation of the micro refinery and the processing of the materials. The controller 151 can transmit micro refinery operating information through the transceiver 197, such as the detected sensor and production information through a computer network to various other computers or servers 223. The controller 151 can also receive control instructions from the other computers and servers 223 for the operation of the micro refinery based upon the detected operating conditions.

The transceivers 197 allow the system controllers 151 to transmit detected operating information such as sensor output data and receive operating instructions from outside sources. The transceiver can be a wired or wireless unit that communicates with a personal computer or a network coupled to other computers 223. A hardwired communications cable can also be run between the operator's computer 223 and one or more micro refineries 101. The communications between the micro refinery 101 and the computers 223 or network can be through various different modes including: radio frequency such as: WiFi, cellular or satellite or optical, such as IR. For example, the type of transmission can be based upon the location of the micro refinery. An operator computer 223 that is close to a private residence or commercial building can have a WiFi communications system. A micro refinery that is beyond radio communications range from a user's computer 223 may require a cellular or satellite communications system.

The micro refinery system information detected by the sensors can transmitted to the microprocessor 601 and stored in memory and/or can then be forwarded to the transceiver and transmitted to a computer network such as the internet. The type of transmission can be based upon the location of the micro refinery. A computer 223 that is close to a private residence or commercial building can have a WiFi communications system. In contrast, a computer 223 that is located in a more remote area may require a cellular or satellite communications system or may require multiple computers 223 and multiple communication paths such as wireless and wired networks.

With the communications systems, the micro refineries can transmit and receive information with a user's personal computer, a system maintenance service, a group administrator, a service group, a supplies provider, an administrative agency or other micro-refineries. In some cases the information can be transmitted from the micro refinery to the user's personal computer. The user can interpret the information and transmit information from the personal computer to servers or other computers. By adding a communication network, utilizing either wired LAN, wireless network such as WiFi, cellular or satellite connection remote communication is possible and the micro refinery can be monitored remotely. The information transmitted over the communications system can be used for various purposes including: system operation monitoring, diagnostics, operation optimization, ethanol production accounting for Government agencies, material reserve monitoring and material ordering, etc. The data transmitted can depend upon the recipient of the information. For example, a regulatory agency may only need to know the ethanol output from the micro refinery. In contrast, a user or maintenance agency will need to know the operating conditions and any processing errors. Thus, more sensor information would normally be transmitted to the user or maintenance agency than a government agency.

Communication systems for micro refineries can also be used for other purposes. For example, in an embodiment the communications system can be used with a carbon credit program discussed in co-pending U.S. patent application Ser. No. 12/110,242, “Method For Using Carbon Credits With Micro Refineries” which is incorporated by reference. The carbon credit program can issue coupons that have a value based upon the reduction of CO₂ emissions caused by using the ethanol produced by the micro refinery instead of the same or equivalent volume of gasoline. The carbon credits can have a value based upon the reduction of CO₂ emissions assuming that the ethanol produced by the micro refinery is used instead of the same or equivalent volume of gasoline. Since the emission of CO₂ from ethanol v. gasoline is directly quantifiable, the reduction in CO₂ emissions can be accurately determined by a sensor that measures the volume of ethanol flowing into the storage tank 145. The system can automatically transmit a volumetric measurement of ethanol produced by the micro refinery so that the corresponding carbon credits can be recorded for the owner of the micro refinery.

The communications system can also be used to trade the carbon credits. Carbon credit buyers may be individuals or corporate entities that may wish to improve the world by reducing carbon footprints. The buys may also need to reduce carbon emissions in order to comply with the mandated government regulations limiting carbon emissions. The carbon credits can be sold to buyers electronically through a computer network or internet based sales system. The micro refinery may indicate the quantity of carbon credits available and sell one or all of the carbon credits at a specified price. Alternatively, the micro refinery may utilize an auction web site to auction some or all of the carbon credits to the highest bidder. In some cases a micro refinery may only have a small quantity of carbon credits and a number of micro refineries can combine and sell their cumulative carbon credits together.

In a preferred embodiment, the micro refinery transceiver can communicate through a network such as the internet with a user computer and/or other server computers that can assist in the monitoring and maintenance of the micro refinery. The micro refinery can share operating information with a user's personal computer and based upon data from the micro refinery, the user can order feedstock, perform maintenance and be notified of any system malfunctions. The recipient of the micro refinery information can occur based upon the type of data being produced. The user's computer can forward operating information to various other entities such as: system maintenance service groups, micro refinery group administrators, micro refinery supply providers and government administrative agencies. In some cases, the micro refineries may share information. For example, micro refineries located in the same area can compare ethanol production output to help identify the optimum operating conditions for the region. The micro refineries in the same area may also share supply information so that larger quantities of supplies can be ordered together and shipping costs can be shared amongst the area's micro-refineries.

For proper operation, feedstock may need to be constantly or periodically input into the micro refinery. By using force sensors in the feedstock storage area, the weight of the supply of feedstock can be monitored. The system can be configured to automatically fill the processing tank with feedstock as it is consumed. The system can also detect the weight/quantity of the feedstock and other batch components or additives in the storage area. When the feedstock or batch component supply in the storage area runs low, the system can inform the user, system maintenance service providers or supply distributors that the batch materials needs to be replenished.

Under perfect processing conditions, the batch may not require constant rebalancing of the chemical components. However, if there are variations in the processing conditions or the micro refinery is simultaneously used to process the batch and discarded alcoholic beverages, the system may need to test and adjust the batch contents of the fermentation tank continuously. Alcoholic beverages with high sugar content may provide sugar to the batch. The required feedstock may also vary depending upon the quantity of ethanol produced from discarded alcoholic beverages. If more ethanol is produced from alcoholic beverages, less feedstock is required to produce the required volume of ethanol.

In yet another embodiment, the system can include sensors that detect the quantity of processed ethanol in the storage tank. If the storage tank is nearly full, the system can reduce ethanol production and if the storage tank is full, the system can stop ethanol production. Conversely, if the system detects that the ethanol storage tank is nearly empty, the system can increase production. The production can be controlled by slowing or stopping the distillation processing. By monitoring the user's ethanol demand and the batch contents, the system will be able to anticipate the need for materials and have the required materials available for processing. The micro refinery system will not have to stop production because the required materials will always be present on site when needed.

The feedstock notification can be transmitted to the user through various communications means including: an electronic signal transmitted from the micro refinery through a network to the user's computer that is displayed as an indicator on a graphical user interface signal. Alternatively, the feedstock notification can be transmitted as an e-mail notification, text or voice message to a user's computer or phone. In other embodiments, the user may authorize or configure the system to automatically contact a feedstock provider and request additional feedstock when the feedstock supplies are low. The signal can be transmitted when there is a predetermined amount of feedstock left in storage, so that the user will have some time to replenish the supply before the system runs out of materials.

In other embodiments, the feedstock deliveries are scheduled and provided on a regular basis. The system can detect the production requirements or the rate of feedstock consumption and adjust the orders to compensate for more or less than expected ethanol production. For example, the production requirements can be based upon the volume of ethanol stored within the system. The system can automatically increase feedstock orders when the reserves are low and decrease the feedstock orders when the storage volume is nearly full. The quantity of the feedstock orders can also be adjusted based upon the projected rate of consumption.

In an embodiment, the system can also be used to detect operating errors in the micro refinery. Field problems can cause equipment downtime and costly onsite service repairs due to equipment malfunctions and extreme weather conditions. To detect equipment problems, the micro refinery may include many sensors that detect the system malfunctions. With reference to FIG. 13, by remotely monitoring the micro refinery and the environment conditions, the system can inform the user computer 223 when there is a problem with the system. The operator can then inspect the system and may be able to fix the problem. If the user cannot fix the problem, the user can call a service center and a repair person can be sent to the micro refinery 101. In some cases, the micro refinery 101 can perform a self diagnostic and determine the problem. This diagnostic information can be transmitted to the user computer 223, service server computer 223 and/or service center 223.

In some installations, the user may not wish to manage the operations of the micro refinery 101 and instead have a service provider maintain the micro refinery. All information can be transmitted to the system maintenance service server 223 or to a micro refinery group administrator computer 223. The user computer 223 may receive basic information such as status from the micro refinery 101 and service updates from the service provider 223 in a graphical format that is easily interpreted by the user. Thus, the micro refinery 101 can be configured to transmit different information in different formats to different computers 223.

In an embodiment, the micro refinery owners can provide access rights to a maintenance company for remote system assistance and sensor data from the micro refinery 101 can be transmitted to a maintenance company server 223 that monitors the operations of the micro refineries 101. Since the maintenance company may not be interested in the appearance of the display, the data output from the micro refinery 101 can be in a “service output mode” which can be primarily raw data with minimal or no data formatting. This raw data reduces the size of the data transmissions which enables many micro refineries 101 to be remotely monitored by a maintenance company. If a problem is detected with any of the micro refineries 101, the maintenance company server 223 can provide digital instructions to the micro refinery 101 or if the problem requires manual adjustments a service technician can go to the micro refinery 101 for servicing or maintenance.

In an embodiment, the micro refinery 101 can be programmed to transmit basic service requests to a user computer 223 and if the problem requires a trained technician, the micro refinery 101 can contact the service center computer 223 so that a technician can be requested immediately. The micro refinery 101 may also be able to identify components that may have failed or be defective and request the required components from the service center computer 223.

In an embodiment, some of the system maintenance can be performed automatically. For example with reference to FIG. 1, in an embodiment, the micro refinery may have a back up porous membrane 135 which is used to separate the ethanol from water. If a porous membrane is damaged, the micro refinery 101 can detect the problem and then automatically switch the valve 161 at the outlet of the distillation tube to a back up porous membrane 135 so that ethanol production can continue. The micro refinery 101 can then request service to repair the damaged or defective porous membrane 135. Various other system components can have a back up unit that can automatically be used in case of failure.

As the number of micro refineries operated by companies and individuals increases, government or regulatory agency monitoring of ethanol production may become necessary. Under commercial fuel regulations, government regulator agencies are allowed access to refinery production information and commercial fuel production companies can be required by law to report fuel sales to government agencies for tax revenues. A similar fuel production reporting may become a requirement for privately owned and operated micro-refineries. However, because the evolution of private micro refineries may be spread throughout the world in various remote locations, it will be difficult for government agencies to monitor personal fuel production using prior methods designed for monitoring large refineries.

Many micro refinery systems may be owned and operated by private individuals who may not know how to report the ethanol produced by their systems. In order to automate the distribution of required information to regulatory agencies, the micro refineries may be configured to automatically transmit production information to an agency accounting system periodically. In other embodiments, the micro refinery may store the production information and the regulator agency may be able to access the production information. By coupling the micro refinery controls to a computer network, the production information can be easily transmitted to any regulatory entity that requires this information. By transmitting the micro refinery data electronically, the user does not have to manually obtain and transmit the ethanol production information to the regulator agency. By allowing electronic communications with the micro refinery, the administrative costs for monitoring ethanol production of the micro refineries are also minimized.

In an embodiment, the ethanol production can be monitored by a fuel flow sensor in the micro refinery 101. The micro refinery 101 can be coupled via remote broadband interface to the associated government agency's computer 223 which can monitor personal fuel production and/or consumption. The data can be transmitted with identification information so that the government can associate the ethanol production information with the proper micro refinery. If the ethanol production from micro refineries 101 is taxed, this tax revenue can be an advantage to local, state and federal government agencies.

In an embodiment, the micro refinery 101 may also include remote control features. In this embodiment, the system includes a plurality of actuators which control the operation of the micro refineries 101. The remote control may be accessible to the user as well as a regulatory agency. If the agency determines that there has been a government regulation violation, the micro refinery 101 in violation can be shut down remotely by the authorized government agency. For example, the regulatory agency may have remote control of an actuated valve that controls the flow of ethanol from the micro refinery 101. By shutting off the valve, the system can be able to process the ethanol that is in the tank but the ethanol in the storage tank cannot be removed from the micro refinery 101. In other embodiments, the government control of the micro refinery can include a progression of activities including initially transmitting messages to the micro refinery owner and/or owner computer 223 regarding the compliance requirements. If the regulatory compliance is not corrected within a predetermined period of time, the regulatory agency can begin to control the operation of the micro refinery 101 and may eventually terminate the micro refinery 101 operation if the operator continues to be in violation of the regulatory requirements.

As discussed above, the micro refinery can include many sensors that detect various operating characteristics of the micro refinery. By monitoring the sensor data and making adjustments based upon the sensor data, the micro refinery can be properly maintained and operated at optimum efficiency. One of the primary factors that will affect the fermentation process is the temperature of the batch. In an embodiment, the temperature of the batch is monitored by a temperature transducer that is coupled to the controller. With reference to FIG. 1, the controller controls the electrical energy applied to the thermoelectric mechanisms 113 and regulates the temperature of the batch using the thermoelectric mechanisms 113 so that the batch temperature is maintained within an optimum temperature range. The system may maintain the batch at a set temperature within a couple of degrees range of a set temperature that can be between about 25° C. to 30° C. The temperature can be most critical during fermentation. After the fermentation process is complete, the system may allow the ethanol temperature to vary with the atmospheric conditions. This allows the system to conserve energy when heating or cooling of the batch is no longer necessary.

In an embodiment, the system can also monitor the fermentation progress by comparing the detected weight of the batch to an expected weight based upon the fermentation time. Ideally, the change in weight should be predictable and within an expected range over time during fermentation. Deviations from the expected rate of weight loss, can indicate a potential fermentation processing problem. For example, a higher than expected weight loss at a specific point during fermentation can be caused by water loss from the fermentation tank due to a leak or evaporation. If the weight loss is lower than expected, this may indicate that there is a problem with the fermentation process. If the batch rate of weight loss is improper, the system can perform additional tests to determine if there is a problem with the batch. In an embodiment, a sample of the batch can be tested to determine if the yeast and other batch components are in the proper concentrations and the sugar is being processed.

In an embodiment, the micro refinery can detect the chemical components within the batch mixture and the system controller may also provide feedback and make corrections if a chemical imbalance is detected. The chemical detection can be particularly useful when a user mixes discarded alcoholic beverages or new feedstock that contain unbalanced ingredient mixtures for maximize ethanol output. For example, if a user processes discarded beer that contains high amounts yeast and no sugar. The lack of sugar can prevent the rate of alcohol fermentation from rising. The lack of sugar can also prevent algae and microbe cellulosic material growth that are used for ethanol processing. The system can detect these chemical imbalances and make the required corrections. In another example, if the system controller detects that too much sugar was added, the system may compensate for this error by increasing the yeast and feedstock needed to process the excess sugar. The micro refinery can include sensors to measure ratios of water, alcohol, sugar, yeast and other chemicals so the proper ingredients can be added to maximize the ethanol production output.

In order to detect the proper concentration of chemicals, the controller may compare the detected quantities or ratios of chemicals to expected component measurements listed in a look up table stored in an electronic memory. As discussed, the fermentation tank can be coupled to a weight sensor and chemical property sensors. The chemical property sensor can detect a concentration of one or more chemicals in the batch. By knowing the components and quantities of the materials in the batch, the system can determine if the batch is an optimum mixture for maximum ethanol production.

The look up table can be a database of chemical components and their relative concentrations. For example, a batch may start with a batch comprising: 80 to 90% weight of sugar such as glucose, 10 to 20% weight of water and 0.5% weight of yeast such as clostridium sporogenes. As the fermentation processing is performed, the sugar content decreases while the ethanol content increases. The relative weights of the materials will change during the fermentation process. An example of a look up table is illustrated below in table 1. The system may utilize look up tables having various different component values that can depend upon the ambient conditions and/or specific materials being processed. In addition to the materials listed in Table 1, the look up table an also include various other batch processing components such as O₂ and CO₂.

TABLE 1

| Fermentation | Sugar | Acceptable | Water | Acceptable | Yeast | Acceptable | Ethanol | Acceptable |
|---|---|---|---|---|---|---|---|---|
| Percentage | Content | Range | Content | Range | Content | Range | Content | Range |
| 0% | 85% | ±3% | 14.5% | ±5% | 0.5% | ±0.2% | 0% | ±0.2% |
| 20% | 69% | ±3% | 14.5% | ±5% | 0.5% | ±0.2% | 16% | ±4% |
| 40% | 53% | ±3% | 14.5% | ±5% | 0.5% | ±0.2% | 32% | ±8% |
| 60% | 37% | ±3% | 14.5% | ±5% | 0.5% | ±0.2% | 48% | ±12% |
| 80% | 21% | ±3% | 14.5% | ±5% | 0.5% | ±0.2% | 60% | ±16% |
| 100% | ≦5% | ±3% | 14.5% | ±5% | 0.5% | ±0.2% | 76% | ±20% |

The look up table can include the optimum data for the batch characteristic measurements that are detected for the point of time in the progress of the fermentation process. If the proper ratio of chemical components is present in the batch, the system will not alter the batch mixture and will allow it to continue to ferment. However, if the batch is chemically imbalanced, one or more components can be added to the batch to correct the chemical imbalance. As the sugar is converted into ethanol, the ratio of sugar in the batch will decline until it has all been converted into ethanol.

As described above, the production of ethanol also produces CO₂. Thus, the weight of the batch will also decrease as the CO₂ gas is released by the batch and vented into the atmosphere. The batch will preferably ferment at a predicted rate and the rate of fermentation can be detected with sensors. If the rate of fermentation drops before the batch is fully processed, this may indicate that there is a problem with the batch that is preventing fermentation from occurring. The system may respond by checking all operating conditions and transmitting a warning to the system operator. The rate of fermentation will also decrease when the batch fermentation processing is complete. By monitoring the decrease in the sugar content, the CO₂ emissions and the decrease in weight of the batch, the system can identify the status of the batch processing. For example, the system can determine if the batch is at a 10%, 40%, 80% or any completion of processing. In an embodiment, the system may continuously add sugar and water to the batch to maintain a steady batch quantity and maximize the fermentation rate.

Based upon the % of completion of fermentation processing, the look up table can include optimal chemical compositions and physical characteristics for the batch. Because the chemical composition of the batch changes with fermentation, the relative quantities of the batch chemicals will also change over time. The look up table can be stored in memory within the micro refinery or in an operator computer that is connected to the micro refinery through a communications network. The micro refinery can also store various preprogrammed feedstock recipes and the corresponding look up tables to help optimize and control the fermentation process depending upon the ambient conditions that accounts for the average temperature, humidity, etc.

Once the proper recipe and % of fermentation are determined, the corresponding optimum chemical composition and physical properties are found on the look up table. The system can compare the detected chemical composition and the optimum chemical composition. If the proper ratio of chemicals for the current process state is not detected, the system can add any deficient materials to correct the chemical ratio. Conversely, if an excessive concentration of a chemical is detected, one or more other batch components can be added to rebalance the mixture.

In an embodiment, the micro refinery may compare the detected chemical concentration to an optimum number but allow for an acceptable range of deviation from the table number. For example, an acceptable quantity of a chemical component may be a range of the optimum concentration. When the system identifies the chemical components that are outside the acceptable range, the required chemical component(s) to be added to the batch are identified and the quantities of each of the required materials are calculated.

The estimated quantities of the materials to be added can be determined by an algorithm. For example, the quantity of material by weight to add to the batch can be determined by the equation quantity (O)=(ΔC)(W) where ΔC is the difference between the detected chemical concentration and the ideal chemical concentration and W is the weight of the batch. In an example, the look up table specifies that chemical component “A” should be 5% of the batch by weight and the measured quantity of component A is only 4% of the batch and the batch weight is 100 lbs. The quantity Q of component A to be added to the batch is determined by multiplying (AC) by the weight of the batch (W), Q=(5%-4%) (100 lbs)=1 pound of component A should be added to the batch.

This batch correction information can be displayed on a display screen for the system or transmitted to an operator computer and/or service computer. The user can then manually add the required components to the batch or the system may add the required components automatically. As discussed above, in an embodiment the system can include containers for the components and control valves that allow measured quantities of the components to be mixed into the batch automatically. If a component is deficient, the system can automatically add the ingredient to the batch. Similarly, if a concentration of a component is too high, the system can add other ingredients to correct and optimize the batch mixture. The system will continue to monitor the batch chemicals throughout the fermentation processing and determine if the batch needs to be modified.

In an embodiment, the system can be controlled by the user or alternatively, the system can be programmed to operate automatically to make corrections to the material mixture. Since the micro refinery is coupled to a computer network, these recipes can be updated by the remote dashboard communication system or provided through online support assistance. The proper or optimum recipe may vary depending upon the ambient conditions and feedstock ingredients being used. As discussed above, a testing mechanism can be used to detect errors in the water, ethanol, sugar, yeast and other chemicals in the batch. The system can respond by automatically correcting the chemical quantities or ratios by adding one or more of the ingredients to the batch. Alternatively, the system may also respond by transmitting a data signal indicating that the batch materials need to be adjusted. The user can then control the micro refinery controller to make the adjustment to the batch materials.

In order to detect the properties of the batch, the test mechanism 171 illustrated in FIG. 1 can include various sensors that are used to measure specific properties of the batch such as the sugar content of the batch. With reference to FIG. 6, the sugar in the batch can be detected using an electrochemical sensor 505. The sensor 505 is coupled to a fluid container that holds a specific quantity of the batch, a reference electrode 509 and an enzyme electrode 507 containing glucose oxidase. The enzyme is reoxidized on the enzyme electrode 507 with an excess of ferrocyanide ion. The total charge passing through the electrode 507 is measured with the ammeter 509 and the total charge is proportional to the concentration of glucose in the batch. The amount of charge can be measured over a fixed time or alternatively, the total charge can be measured for a completed reaction. The ammeter 509 is coupled to the controller 513 which calculates the concentration of glucose in the batch and compares the detected glucose to the target concentration. The controller 513 can make adjustments to the batch or operating conditions if the concentration of glucose is not within the expected range.

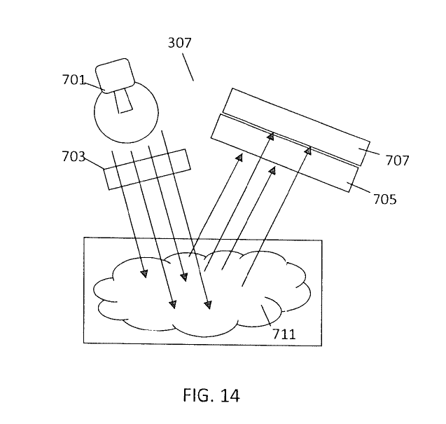

In an embodiment, the detection system can include an optical system which exposes the sample of the batch to light. In an embodiment and with reference to FIG. 14, an optical detection system 307 can include a light source such as a xenon lamp 701, filters 703, 705 and a CCD spectrometer 707. It is possible to monitor both the yeast culture fluorescence and the cell concentration in the batch 711. The transmitted light irradiates the yeast in the batch 711 in front of the CCD spectrometer 707. The fermenting yeast responds to the light by emitting a fluorescence emission that is shifted towards longer wavelengths in comparison to the light source wavelengths. The fluorescent lighting is detected by the CCD spectrometer 707. The short pass filter 703 can allow light transmission in the spectral regions between 300 to 400 nm, which can be the fluorescence excitation, and 700 to 850 nm which can be the light scattering signal. The filters 703, 705 can also block the output of the xenon lamp in the region between 400 to 700 nm to avoid an interference with the fluorescence emission. By detecting the fluorescence and intensity of the fluorescence, the CCD spectrometer 707 can detect the fermentation processing of the yeast.

The batch testing mechanism can also include chemical detection system which can determine the chemical components in the batch. In an embodiment, absorption spectroscopy is used to detect the substances present in a sample, which can be a solid, liquid, or gas, and subsequent promotion of electrons from one energy level to another in that substance. The wavelength at which the incident photon is absorbed is determined by the differences in the energy levels of the different substances present in the batch sample. Various wavelengths of light can be used to detect the chemical composition. In absorption spectroscopy, the absorbed photons are not re-emitted by the batch and the absorbed energy is transferred to the chemical compound upon absorbance of a photon.

At a given wavelength, the measured absorbance has been shown to be proportional to the molar concentration of the light absorbing batch component. The amount of radiation absorbed versus wavelength for a particular compound is known as the “absorption spectrum.” At wavelengths corresponding to the resonant energy levels of the sample, some of the incident photons are absorbed, resulting in a drop in the measured transmission intensity and a corresponding dip in the spectrum. The absorption spectrum can be measured using a spectrometer and by knowing the shape of the spectrum, the optical path length and the amount of radiation absorbed. By analyzing the absorption spectrum, the chemical components and the concentrations of the compounds within the batch can be determined.

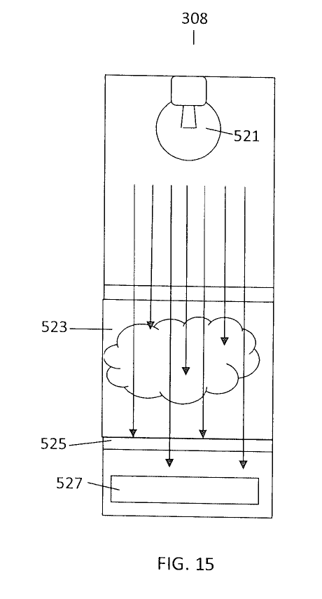

With reference to FIG. 15, the main components of the gas sensor 308 are an infrared light source 521, a sample chamber or light tube 523, a wavelength filter 525, and the light detector 527 such as a CCD spectrometer. The gas enters the sample chamber 523 and the concentration of the target component is measured electro-optically by its absorption of a specific wavelength in the infrared spectrum. The light 521 is directed through the sample chamber 523 towards the detector 527. An optical filter 525 can be mounted above the detector 527 that eliminates all light except the wavelength that the selected gas molecules can absorb. The detected concentration of target component can be compared to a look up table value of target component to determine the status of the batch materials. Based upon the detected target component, the system can made adjustments to the batch processing as needed. In addition to target component detection, the optical system can also monitor the presence of other chemical components within the batch that absorb different wavelengths of light. The system can monitor the detection of these other wavelengths or use separate optical sensors configured to detect the relative concentrations of other chemicals in the batch. In an embodiment, the fermentation of the batch can be detected by the production of CO₂. Since CO₂ is produced during fermentation, the quantity of CO₂ indicates the rate of fermentation and when the emission of CO₂ stops fermentation has also stopped.

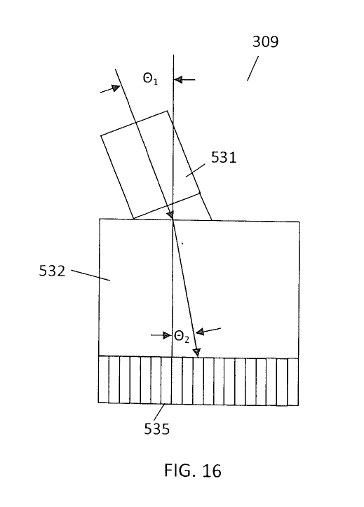

In another embodiment, the presence of a chemical component in the batch can be detected by a sensor that measures the refraction of light through the batch liquid. With reference to FIG. 16, a refractive detection system 309 is illustrated that includes a light source 531, a fluid chamber 532 and an array of light sensors 535. A piece of glass can separate the light source 531 from the liquid 501 while allowing light to pass through the fluid chamber 532. The light beam contacts the intersection of the glass and fluid at a first fixed angle, θ₁ and the light beam is refracted at the intersection causing the light beam to bend to a second angle, θ₂. The array of sensors 535 is arranged on the opposite side of the fluid chamber 523 in a linear configuration to detect the angle of refraction, θ₂. Light refraction is described by Snell's law, which states that the angle of incidence is related to the angle of refraction by sin θ₁/sin θ₂=v₁/v₁=n₁/n₂ or n₁ sin θ₁=n₂ sin θ₂. The variables in these equations are defined as follows: v₁ and v₂ are the wave velocities through the respective media, θ₁ and θ₂ are the angles between the normal (to the interface) plane and the incident waves respectively and n₁ and n₂ are the refractive indices.

Different fluids can have different refractive indexes. Thus, a fluid containing certain chemical components can have an index of refraction that is different than a fluid that does not have the chemical components. In this embodiment, the system will detect the proper chemical combination based upon the detected index of refraction. If the angle of refraction is not within the required range, the system will cause the flow controller to prevent fluid 501 from flowing from the storage container. Because the angle of the refracted light must be precisely detected, a laser light source may be used to measure the refraction angle.

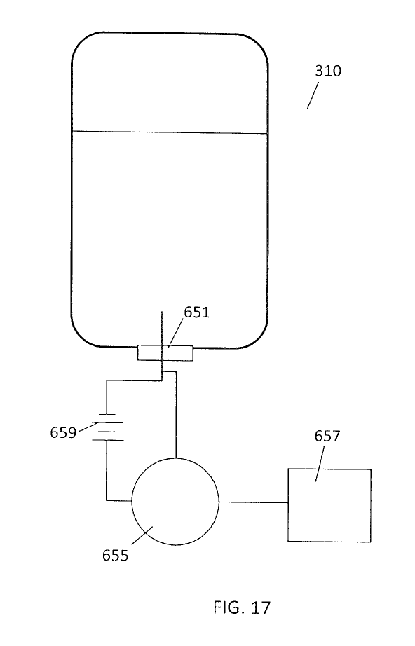

In an embodiment, the test mechanism of the micro refinery can also detect the pH level of the batch and add chemicals to the batch to keep the pH level between about 4.0-5.0 which can be an optimum pH level for fermentation. With reference to FIG. 17, in an embodiment a pH sensor 310 may be used to detect the pH level of the batch. The pH sensor can comprise a pH electrode 651 sealed to an insulating tube containing a solution having a fixed pH value that is in contact with a silver-silver chloride half cell. The potential developed across the membrane is compared to a stable reference potential. The pH electrode 651 has a porous constriction which allows the batch fluid to flow into the test sample area. A power supply 659 and a voltmeter 655 are coupled to the pH electrode 651. The pH electrode 651 is exposed to the batch liquids and the electrical resistance is proportional to the pH level. The output of the pH electrode 651 is detected by the volt meter 655.

The pH level is defined as pH=−log(H+) where H+ is the hydrogen concentration in solution, at normal room temperatures and provides an indication of whether a batch is acidic or basic. The pH scale ranges from 0 (acid) to 14 (alkaline). The output of the pH sensor is coupled to a controller 657 which compares the detected pH value for the batch to a target pH value. In an embodiment, the batch may have a target pH value of 4-5 or the target value can be determined based upon a look up table stored in memory. If the pH value is outside the target value or range, the system can issue a warning to the operator or automatically adjust the pH level by adding acidic or base materials. The system can continuously monitor the pH level and make adjustments as necessary to keep the pH level within the target range.

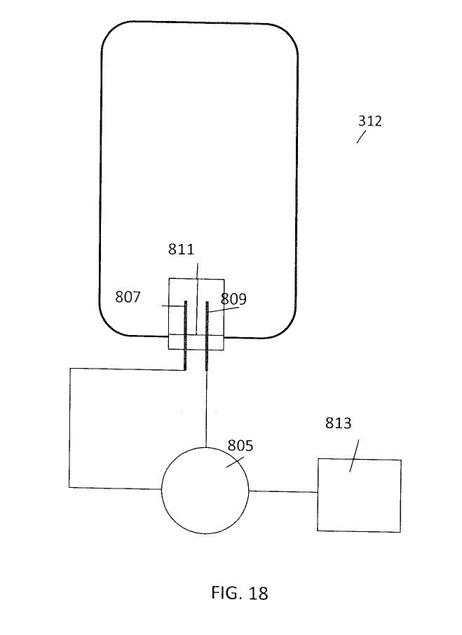

In another embodiment, the testing mechanism can also detect the quantity of dissolved oxygen in the batch. For optimum fermentation, the batch will include a minimal amount of dissolved oxygen. With reference to FIG. 18, an oxygen sensing system 312 is illustrated. An exemplary technique for measuring dissolved oxygen includes a dissolved oxygen sensor 805 that includes an anode 807 and cathode 809 which are immersed in electrolyte within the sensor body. Oxygen enters the sensor through a permeable membrane 811 by diffusion, and is reduced at the cathode 809 to create a measurable electrical current. The cathode 809 is a hydrogen electrode and carries a negative potential with respect to the anode 807. The oxygen permeable membrane 811 also separates the anode 807 and cathode 809 from the batch liquid being measured and oxygen diffuses across the membrane 811. With no oxygen, the cathode 809 becomes polarized with hydrogen and resists the flow of current. When oxygen passes through the membrane, the cathode 809 is depolarized and electrons are consumed. The cathode 809 electrochemically reduces the oxygen to hydroxyl ions and the anode 807 reacts with the product of the depolarization with a corresponding release of electrons. The oxygen interacts with the internal components of the probe to produce an electrical current. The output from the dissolved oxygen sensor can be a variable electrical voltage in millivolts and a current flowing through the circuit is detected by an ammeter 805. The electrode pair 807, 811 permit current to flow in direct proportion to the amount of oxygen entering the sensor. The magnitude of the current is a direct measure of the amount of oxygen entering the probe. There is a linear relationship between the oxygen concentration and the electrical current. The output from the ammeter 805 is coupled to a controller 813 that calculates the dissolved oxygen level based upon the detected current.

The quantity of dissolved oxygen in the batch can affect the rate of fermentation. In an embodiment, a dissolved oxygen level of 10% or below is acceptable but higher levels for example, 20% will reduce the fermentation rate. The dissolved oxygen can be controlled by the aeration and agitation of the batch. By submerging the agitator within the batch and decreasing the rotational speed, the dissolved oxygen level can be reduced. In an embodiment, the system can monitor the dissolved oxygen levels and control the dissolved oxygen level of the batch within a predetermined range by adjusting the agitator position and velocity.

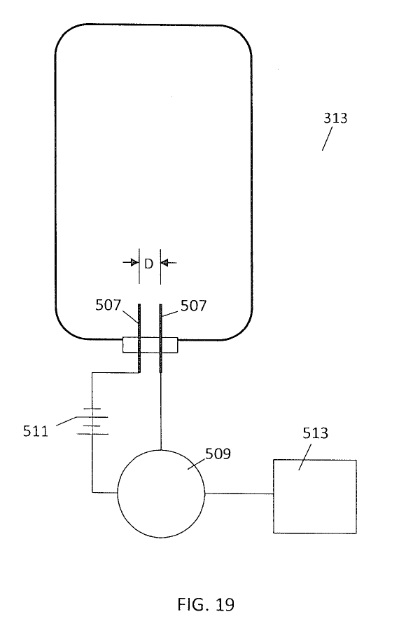

The test mechanism can also measure the electrical resistivity of the batch. The electrolytes in the batch reduce the electrical resistance. In an embodiment, the batch includes a specific concentration of electrolytes. With reference to FIG. 19, the resistivity sensor 313 is in contact with a test container 503 that holds a sample 501 of the batch that includes two electrodes 507 that have a known length and are positioned in parallel and separated by a known distance “D.” An electrical voltage 511 is applied to the electrodes 507 and the resistivity of the sample of the batch material between the electrodes 507 is measured. A high purity batch fluid solution may have a high electrical resistivity. However, when a salt, like sodium chloride is dissolved in water the sodium and chloride separate temporarily. The sodium will become a positively charged ions and the chloride will become a negatively charged ions. The ions behave as electrolytes when dissolved in water and cause the batch fluid to be more conductive. Thus, when a voltage is applied to the sensor electrodes 507, the resistivity of the batch sample 501 is inversely proportional to the concentration of electrolytes. A higher concentration will decrease the electrical resistivity. The relationship between the voltage and resistivity is (R) resistance=(V) voltage/(1) current. The sensor can include two electrodes 507 with a fixed voltage applied across the leads. The electrodes are made from electrically conductive materials such as metals and should be corrosion resistive. The resistance of the batch can be determined by measuring the current flowing between the electrodes with an ammeter 509. The ammeter 509 can be coupled to the controller 513 which can add electrolytes to adjust the resistivity of the batch. For example, if the detected resistivity of the batch is within the target range, the controller 513 will allow the micro refinery to continue to process the batch. In contrast, if the test sample of the batch has an electrical resistivity higher than the optimum range, the controller 513 can respond by adding electrolytes to the batch. If the measured electrical resistivity is below the optimum range, this can be an indication that the batch contains excessive electrolytes, salts or other impurities which may reduce the fermentation rate. The system can respond to a low resistivity measurement by completing the fermentation processing of the batch and then cleaning the fermentation tank to remove all impurities before performing any additional fermentation processing. In an embodiment, the look up table can include optimum values for some or all of the sensor data described above. The system can then monitor the sensor data and compare the sensed data to the look up tables to monitor the fermentation processing. Because there can be a substantial amount of data, the sensors can be configured as part of a neural network to analyze and predict the batch fermentation processing.

In addition, sensors for testing of the batch in the fermentation tank, the micro refinery can also include one or more sensors that detect the presence of water mixed with the ethanol at the outlet of the membrane separation system and/or in the storage tank. The sensors are coupled to the controller that monitors the water content. If the maximum water content is exceeded, the micro refinery controller can transmit a warning signal. The presence of water in the ethanol can be problematic when used as a fuel for internal combustion engines. In an embodiment, the micro refinery can detect the water of the ethanol in the storage tank and process the ethanol in a manner that accounts for the water when blending the ethanol with gasoline before being pumped into a vehicle. In an embodiment, the maximum ratio of ethanol to gasoline can be based upon the percentage of water detected in the ethanol. If a high percentage of water is detected in the ethanol, a lower percentage of ethanol is mixed with the gasoline to minimize the total percentage of water in the fuel tank. Alternatively, the micro refinery can perform additional processing to remove the water from the ethanol through additional filtration. Conversely, if a very low percentage of water is detected a higher concentration of ethanol can be mixed with the gasoline to prevent excess water from being used.

As discussed above, the micro refinery can transmit data to other computers through a network. The sensor data detected by the micro refinery can be processed and output in various different ways. The micro refinery has been described as performing most of the operating data analysis so that the signals output to the computer network include status information or requests for feedstock or repair/maintenance. In an embodiment, the system can transmit all of the raw sensor data through a network to a remote maintenance computer that runs software for operating the micro refinery. The maintenance computer can analyze the detected data and perform some or all of the calculations to determine the status of the different systems and transmit the requests for feedstock or repair/maintenance. The maintenance computer can then display the status or requests for feedstock or repair/maintenance.

In an embodiment, the system can display the sensor information in different ways according to who the viewer is. The raw sensor data can be cumbersome for a user to monitor and understand. However, for a highly trained technician, a visual display that includes all of the raw data can be the best means for troubleshooting a micro refinery system. The sensor output can also be displayed in a graphical “dashboard” user mode. The user mode can allow the system to display the detected sensor information in a manner that provides the basic operating status information in a simplified format that is easily understood by a user. This graphical dashboard can be output to the internet so that the micro refinery can be can be monitored online anywhere in the world. The user mode graphical dashboard data can include: feedstock level, water level, ethanol level, fermentation time table for completion, future appointment schedule and feedstock delivery date or service repair date.

It will be understood that the inventive system has been described with reference to particular embodiments, however additions, deletions and changes could be made to these embodiments without departing from the scope of the inventive system. For example, the same processes described can also be applied to other devices. Although the systems that have been described include various components, it is well understood that these components and the described configuration can be modified and rearranged in various other configurations.

What is claimed is:

1. A micro refinery apparatus comprising:
a fermentation tank for fermenting a batch that includes water, sugar and yeast;
a distillation tube having multiple sections that is coupled to the fermentation tank for distilling ethanol;
a coupling mechanism for releasably securing the multiple sections of the distillation tube together;
a gimbal mechanism coupled to the distillation tube for aligning of the distillation tube in a vertical orientation;
a filtration mechanism that is coupled to the distillation tube for separating the ethanol from water; and
a storage tank coupled to the filtration mechanism for storing the ethanol.

2. The apparatus of claim 1 wherein the coupling mechanism includes a plurality of pins and hooks that are secured around an outer diameter of the distillation tube and engage each other to couple the sections of the distillation tube and disengage to release the sections of the distillation tube.

3. The apparatus of claim 1 further comprising:
a plurality of perforated plates vertically spaced apart and substantially parallel to each other and mounted in a diagonal orientation within the distillation tube.

4. The apparatus of claim 3 further comprising:
a plate assembly coupled to the plurality of perforated plates;
wherein the plate assembly is mounted within the distillation tube.

5. The apparatus of claim 1 further comprising:
a gyroscope coupled to the distillation tube for maintaining a vertical alignment of the distillation tube.

6. The apparatus of claim 5 wherein the axis of rotation of a rotating rotor within the gyroscope is aligned with a vertical axis of the distillation tube.

7. The apparatus of claim 1 further comprising:
a porous membrane coupled to the filtration mechanism having a plurality of pores that are larger than water vapor molecules but smaller than ethanol vapor molecules.

8. The apparatus of claim 7 wherein the pores of the porous membrane are between about 3 to 30 angstroms in diameter.

9. A micro refinery apparatus comprising:
a fermentation tank for fermenting a batch that includes water, sugar and yeast;
a distillation tube coupled to the fermentation tank for distilling ethanol;
an alignment mechanism coupled to the distillation tube for maintaining a vertical alignment of the distillation tube;
a filtration mechanism that is coupled to the distillation tube for separating the ethanol from water;
a storage tank coupled to the filtration mechanism for storing the ethanol;
a sensor that detects a physical property of the batch during the fermenting step;
a CPU that receives a signal from the sensor representing the physical property of the batch and determines if the physical property of the batch is outside the acceptable range and transmits a warning signal when the physical property is outside the acceptable range.

10. The apparatus of claim 9 wherein the distillation tube has multiple sections and a coupling mechanism is used for releasably securing the multiple sections of the distillation tube together.

11. The apparatus of claim 10 wherein the coupling mechanism includes a plurality of pins and hooks that are secured around an outer diameter of the distillation tube and engage each other to couple the sections of the distillation tube and disengage to release the sections of the distillation tube.

12. The apparatus of claim 9 further comprising:
a plurality of perforated plates vertically spaced apart and substantially parallel to each other and mounted in a diagonal orientation within the distillation tube.

13. The apparatus of claim 12 further comprising:
a plate assembly coupled to the plurality of perforated plates;
wherein the plate assembly is mounted within the distillation tube.

14. The apparatus of claim 9 further comprising:
a gyroscope coupled to the distillation tube for maintaining a vertical alignment of the distillation tube.

15. The apparatus of claim 9 wherein the axis of rotation of a rotating rotor within the gyroscope is aligned with a vertical axis of the distillation tube.

16. The apparatus of claim 9 wherein the CPU compares the physical property to a look up table that includes an optimum range of the physical property.

17. The apparatus of claim 9 further comprising:
a porous membrane coupled to the filtration mechanism having a plurality of pores that are larger than water vapor molecules but smaller than ethanol vapor molecules.

18. The apparatus of claim 14 wherein the pores of the porous membrane are between about 3 to 30 angstroms in diameter.

19. The apparatus of claim 18, further comprising:
a first storage bin for storing the first chemical component; and
a first valve controlled by the CPU and mounted between the first storage bin and the fermentation tank;
wherein the physical property is a concentration of a first chemical in the batch and if the concentration of the first chemical component is outside the acceptable range, the CPU opens the first valve to add the first chemical component to the fermentation tank.

20. The apparatus of claim 9 further comprising;
a thermoelectric mechanism having two metal plates mounted on opposite sides of a ceramic core coupled to the fermentation tank for heating and cooling the batch.
